(19) United States
(12) Patent Application Publication
(10) Pub. No.: US 2022/0142653 A1
(43) Pub. Date: May 12, 2022

(54) TRANSCERVICAL ACCESS SYSTEMS FOR INTRAUTERINE FLUID EXCHANGE, SUCH AS PLACEMENT OF HYDROGELS FORMED IN SITU

(71) Applicant: Pramand LLC, Bedford, MA (US)

(72) Inventors: Michael Bassett, Hampton, NH (US); Ian Feldberg, Sudbury, MA (US)

(21) Appl. No.: 17/494,752

(22) Filed: Oct. 5, 2021

Related U.S. Application Data
(60) Provisional application No. 63/113,013, filed on Nov. 12, 2020.

(51) Int. Cl. A61B 17/12; A61M 25/00; A61M 5/19; A61M 5/315; A61B 17/42

(52) U.S. Cl. CPC A61B 17/12181; A61M 25/0082; A61B 17/12099; A61M 2210/1433; A61M 5/31596; A61B 17/42; A61M 5/19

(57) ABSTRACT

Transcervical access systems for providing transcervical movement of fluids are described. The transcervical access systems are effective for transferring a broad range of fluids, including the delivery of hydrogel precursors, saline, and imaging fluids, to the uterine cavity. The transcervical access systems are also effective for removing fluids from the uterine cavity, such as residual bodily fluids, residual fluids from a procedure, or tissue. The transcervical access systems described include flow limiters, such as egress limiters and/or cervical plugs. Methods of use of the transcervical access systems are also described. Methods include using the transcervical access systems to transcervical access the uterine cavity and install hydrogel. The transcervical access systems and associated methods can be useful for providing degradable hydrogel in the uterine cavity, including the cervical canal, for the prevention of adhesions following intrauterine procedures.

### CROSS REFERENCE TO RELATED APPLICATIONS

This application claims priority to copending U.S. provisional patent application 63/113,013 filed on Nov. 12, 2020 to Bassett et al., entitled “Placement of Hydrogels Formed In Situ, Composition Design and Delivery Tools for Intrauterine Use,” incorporated herein by reference.

### FIELD OF USE

Aspects of the invention relate to methods of applying materials delivered to the uterine cavity, including in situ forming hydrogels, and to tools useful for the placement, instrumentation, and delivery of these materials.

### BACKGROUND

The unwanted adherence of scar tissue that can occur following intrauterine procedures, known as intrauterine adhesions typically occurs when two injured tissue surfaces are close to one another. This complication can lead to painful and debilitating medical problems, including but not limited to postoperative adverse events, failure of the medical intervention, and infertility. Surgical dissection and resection of adhesions often result in a high rate of adhesion re-formation. Current methods for preventing adhesions, including but not limited to, intrauterine adhesions, have limited effectiveness.

### SUMMARY OF INVENTION

Methods and devices for delivering or removing fluid with respect to the uterine cavity, which in some embodiments involves in situ forming hydrogels to prevent the formation of intrauterine adhesions, are presented herein. In situ forming hydrogel technologies may also be used as a tamponade to stop unwanted bleeding post-surgery and to provide mechanical support for uterine tissues. Materials may be introduced into a surgical site to reduce or prevent contact between damaged tissues, or portions of the tissues. Flowable components may be used so as to ease the introduction and formation of the materials. For example, flowable polymer precursors may be introduced in a transcervical manner and activated to form a material in the uterus after its introduction. Examples of precursors include polymerizable, crosslinkable, and thermosetting polymers that form a material, e.g., a hydrogel, inside the uterus. In some embodiments, other fluids for treatment, imaging or other purpose can be delivered using the improved catheter system described herein. The catheter system can also be used to remove fluid from the uterine cavity, such as to prepare the patient for further procedures or to collect materials for biopsy. Fluids can be considered broadly as materials that can flow into and/or out from the catheter of the device and may include tissue elements suitable for tissue biopsy.

Some embodiments relate to a method of preventing adhesion of damaged tissue surfaces in a potential space, such as a uterus. The method comprising introducing a flowable material into a uterus to tamponade a surface within the uterus. The tamponade can be effective to reduce bleeding from damaged tissues after surgical procedures. The material may be, e.g., a hydrogel and may function as a stent or a splint. Some embodiments relate to a method of preventing adhesion in a uterus by application of a material crosslinking at least one precursor to form a hydrogel in the uterus, e.g., to coat the surface of damaged tissue, or tamponade a surface of the uterine cavity, or to prevent the collapse and adherence of the uterine cavity walls to each other. Additional embodiments relate to the pre mixing of hydrogel constituents into one precursor and the activation of the crosslinking during application with a second precursor. Embodiments relating to the design of the application device include the use of a soft, flexible atraumatic catheter, the addition of side port(s) for improved application to the intrauterine surface, and a low catheter profile for reduction of remaining insertion track after removal. The incorporation of a rounded non-traumatic feature to the outside of the catheter body, in the form of an external plug or balloon feature, that can be adjusted along the catheter length, to be held at the cervical os to control the flow of excess hydrogel egress from the target tissue.

In one aspect, the invention pertains to a crosslinked hydrogel composition comprising multi-armed polyethylene glycol molecules crosslinked with a multifunctional crosslinker having a molecular weight of no more than about 2 kDa with biodegradable crosslinking bond with a swelling of no more than about 125% by weight after 24 hours following placement in a neutral buffered saline solution. In some embodiments, the hydrogel can have an in vivo, intrauterine degradation time from about 3 hours to about 29 days. To provide stability without undesirable pressure on the patient for in vivo use, the hydrogel can have values of Young's modulus from 5 kPa to 300 kPa. In some embodiments, the crosslinking molecule incorporated into the hydrogel is a polyamine, such as polylysine, which can be trilysine. In some embodiments, the crosslinking functional groups are N-hydroxy succinimide esters and primary amines that react to form amide bonds through nucleophilic substitution.

In a further aspect, the invention relates to a method for providing a in situ forming tamponade or adhesion inhibitor into a uterine cavity, the method can comprise delivery of a crosslinking hydrogel precursor solution through an applicator catheter in a configuration to facilitate uniform delivery of the precursor solution into the cavity, in which the hydrogel swells no more than 125% after formation in vivo and degrades in from about 3 hours to about 21 days. In some embodiments, the hydrogel polymer composition comprising multi-armed polyethylene glycol polymer core PEG precursor) crosslinked with a multifunctional crosslinker (crosslinker precursor) having a molecular weight of no more than about 2 kD, and the precursor solution is formed by mixing the PEO precursor, the crosslinker precursor and an accelerating compound in a flow directed to the applicator catheter to initiate the crosslinking reaction. The delivery from the catheter is performed quickly so that the precursor solution entering the cavity has a low enough viscosity to fill the space while crosslinking quickly enough so that the procedure can be completed in a desirable timeframe thus preventing undue loss of the precursors from the uterine cavity. The applicator can comprise or be associated with a flow restrictor that helps to maintain the hydrogel precursor within the uterine cavity such that separation of the uterine walls is achieved by complete filling of the uterine cavity.

In another aspect, the invention relates to a hydrogel applicator that comprises two reservoirs with outlets connected to a Y-connector to intermix the respective solutions from the reservoirs in a section of tubing that connects to a catheter having a size of no more than 9 Fr having an outlet port on the side of the catheter body with an atraumatic tip. The reservoirs can be syringe tubes mounted in a holder the plungers connected to a plunger cap to prove for convenient simultaneous deployment of the two syringe plungers. A static mixer can provide more rapid mixing of the combined solutions from the reservoir. The catheter can be formed from a sufficiently low durometer value polymer so that it is less likely to cause injury to the patient's tissue. In some embodiments, one reservoir can hold a blend of hydrogel precursors if the crosslinking is sufficiently slow, and the other reservoir can then comprise an accelerant, such as a basic buffer.

In an aspect, the invention pertains to a transcervical access system for movement of fluids with easy manipulation, the transcervical access system comprising:

a graspable structure comprising one or more fluid reservoirs and one or more actuators to direct flow from or into the one or more fluid reservoirs;

a catheter comprising a tubular element with a lumen, an outer diameter, an average wall thickness, and one or more distal ports, wherein the catheter engages the graspable structure with a configuration to provide fluid flow through the tubular element of the catheter upon actuation of the actuator; and

an egress limiter comprising a tubular member and a cap element fixedly attached to the tubular member at or near an end the tubular member, the tubular member having an inner lumen with an inner diameter larger than the outer diameter of the tubular element of the catheter such that the egress limiter can slide over the catheter and is removable from the catheter, wherein the tubular member has a length less than the length of the tubular element of the catheter, wherein the position of the tubular member allows for adjustment of a distal catheter length, wherein the distal catheter length comprises a length from the distal end of the catheter to the distal end of the cap element. In some embodiments, the system is suitable for single-handed manipulation.

In some aspects, the invention pertains to a transcervical access system for intrauterine movement of fluids with easy manipulation, the transcervical access system comprising:

a graspable structure comprising one or more reservoirs and one or more actuators to direct flow from or into the one or more fluid reservoirs;

a catheter comprising a tubular element with a lumen, an outer diameter, and one or more distal ports, wherein the catheter engages the graspable structure with a configuration to provide fluid flow through the tubular element of the catheter; and

a cervical plug having an inner lumen with an inner diameter larger than the outer diameter of the tubular element of the catheter such that the cervical plug can slide over the catheter and is removable from the catheter, wherein the cervical plug has an outer diameter suitable for placement in the cervix.

In an additional aspect, the invention pertains to a method for transcervical movement of a fluid into or out from a patient's uterine cavity, the method comprising:

transferring fluid into or out from a patient's uterine cavity using a catheter system comprising:
a graspable structure comprising a reservoir of a hydrogel precursor and an actuator,
a catheter comprising a tubular element with a lumen, an outer diameter and one or more distal outlets, wherein the catheter is connected to the reservoir with a configuration to provide fluid flow through the tubular element of the catheter and wherein the tubular element has a suitable length for transcervical intrauterine delivery,
and
a blocking structure comprising a lumen with an inner diameter larger than the outer diameter of the tubular element of the catheter such that the blocking structure can slide over the catheter, wherein the blocking structure has been positioned to adjust a distal catheter length, wherein the distal catheter length comprises a length from the distal end of the catheter to the distal end of the blocking structure; and

removing the catheter from the patient while leaving the blocking structure in place to block the fluid from exiting the cervix.

### BRIEF DESCRIPTION OF THE DRAWINGS

FIG. 1A is a depiction of a transcervical access system with an egress limiter and a single syringe.

FIG. 1B is a depiction of a transcervical access system with an egress limiter and dual syringes connected to a catheter via a Y-connector.

FIG. 2 is a depiction of the transcervical access system of FIG. 1B in which an optional T-branch connector and syringe are provided.

FIG. 3 is a cross-section of a basic installation tip.

FIG. 4 is an illustration of various installation tip geometries with radially positioned openings along the circumference of the catheter.

FIG. 5 is a depiction of a transcervical access system being used to deliver flowable components to the uterus through the cervix, with the cap element being used to control catheter placement and egress of material from the external cervical os.

FIG. 6 is an illustration of various cap element designs.

FIG. 7 is a depiction of an assembled configuration of a catheter and an egress limiter (A) and a separated configuration of a catheter and an egress limiter (B).

FIG. 8A is an illustration of a catheter assembly with an egress limiter and connector.

FIG. 8B is an enlarged cross-section of a portion of the catheter assembly and egress limiter of FIG. 8A.

FIG. 8C is an exploded view of the catheter assembly, egress limiter, and connector of FIG. 8A.

FIG. 9 is a depiction of a catheter assembly with a luer connector and an egress limiter having an inflatable/deflatable adjustable cap element connected via an internal balloon lumen to an inflation/deflation port.

FIG. 10 is an illustration of a catheter assembly with a cervical plug and a connector.

FIG. 11 is an illustration of a catheter assembly with a cervical plug, an egress limiter, and a connector.

FIG. 12 is an illustration of various cervical plug shapes.

FIG. 13 is a depiction of a transcervical access system with a cap element being used to transcervically deliver flowable components to the uterus.

FIG. 14 is a depiction of a transcervical procedure using a transcervical access system with an egress limiter.

FIG. 15 is a depiction of a transcervical procedure using a transcervical access system with a cervical plug.

FIG. 16 is a depiction of a transcervical procedure using a transcervical access system with a cervical plug and an egress limiter.

FIG. 17 is a depiction of a transcervical procedure using a transcervical access system with a cervical plug having a tapered teather or grip end.

FIG. 18 is a photograph taken after using a standard Cook Goldstein Sonohysterography Catheter to install hydrogel into a human uterus.

FIG. 19 is a series of post peri-hysterectomy pathology photographs taken after using the delivery system to install hydrogel after a uterine ablation procedure. The photographs show a removed uterus, the removed uterus that has been cut open to reveal the installed hydrogel, and the cut open uterus with the excised hydrogel implant.

### DETAILED DESCRIPTION OF THE INVENTION

A catheter system is described to provide for fluid movement into or out from a patient's intrauterine cavity. In embodiments of particular interest, effective approaches are presented for reducing or eliminating uterine adhesions resulting from surgical procedures through the delivery of appropriately designed hydrogel precursors using improved applicators that are engineered to provide stable hydrogel delivery to all relevant locations of the uterus. Effective hydrogel precursors can be designed with respect to one or more of gelation times, viscosities of the precursor solutions, swelling degree after crosslinking, or bio-degradation times. Improvements with respect to these parameters can overcome shortcomings of earlier attempts to deliver useful products for adhesion prevention based on hydrogels. An improved catheter system, which can function as an applicator, is designed for more effective delivery into a cavity, such as a uterine space through delivery while blocking flow out from the cervix, as well as for some embodiments, appropriate mixing of the precursors to control placement of the hydrogel based on the gelation time. The improved applicator also is designed to avoid accidental removal of hydrogel from the cervix when withdrawing the applicator so that adhesions can be inhibited from locations near the cervix. The improved applicator can comprise a temporary cervical cap that remains in place when the catheter is removed from the cervix and/or a cervical plug that remains in lace following the procedure to stabilize the hydrogel in the uterus and inhibit involuntary ejection of hydrogel from the uterus due to natural contractions. A blocking structure can refer to an egress limiter that comprises the cervical cap, or a cervical plug, or both. Corresponding methods based on the use of the improved applicator are described. Through the use of one or more of these improved features, an effective treatment approach can be provided to alleviate a common source of post-procedure complications.

Certain embodiments of the improved applicator are directed to an apparatus for delivering either a single solution or two or more crosslinkable solutions to form hydrogel implants in situ. Based on the design of the applicator, corresponding methods can be effectively performed by a health care professional with straightforward manipulations. Included herein are single-, dual- and multi-component hydrogel systems for such use and delivery systems for depositing such hydrogel systems. Some embodiments involve forming a gel or hydrogel from a precursor, which would be a material that becomes integrated into the gel or hydrogel structure. A monomer or macromer used to form a gel or hydrogel would typically be a precursor, but a polymerization accelerating agent would typically not be considered a precursor, although its presence is directly involved in the hydrogel formation.

While the catheter system can be particularly effective as an applicator for the delivery of hydrogel precursors, the device can also be effective for the delivery of other fluids used for intrauterine imaging, such as but not limited to saline, contrast mediums, and sterile gel preparations. In addition, the catheter system can also be effective to remove fluids from the intrauterine cavity. For example, fluid can be removed prior to infusion of hydrogel to reduce dilution effects. Also, fluid can be removed to capture released tissue cells for performing a biopsy.

### Intrauterine Adhesions

Intrauterine adhesions (IUAs) appear as adhesion bands with clear or irregular margins, which lead to distortions of natural uterine physiology, and ultimately may fill the uterine cavity (1). A partial or total blockage of the uterine cavity due to adhesions may result in abnormal bleeding, infertility, and recurrent pregnancy loss (2). For any of these reasons, avoidance of intrauterine adhesions is desirable. IUAs are commonly found in patients following gynecological procedures involving instrumentation placed in the uterus for either diagnostic or therapeutic purposes, or in patients having experienced trauma within the uterine cavity (3). The incidence of intrauterine adhesion formation following such events can be as high as 60% (4). Adhesions are the results of operative hysteroscopy, with rates varying with the type of procedure involved, and notably high rates in metroplasty, myomectomy, and endometrial ablations (5, 6). In these conditions while treating the primary cause of subfertility, one risks creating adhesions, which present a more insidious risk to fertility. The association between presence of adhesions and infertility has been reported as high as 43% (3). Furthermore, evidence suggests that the severity of adhesions may be progressive with mild, filmy adhesions that may advance to fibromuscular adhesions and ultimately developing into dense connective tissue (8). Various factors have been associated with intrauterine adhesions formation. (6, 9, 10, 11, 12).

The use of resorbable barriers for the prevention of IUAs has shown some clinical success in past years. Barriers include solutions of hyaluronic acid, crosslinked hyaluronic acid and viscoelastic solutions comprising hydrophilic polymers. Solutions of hyaluronic acid and crosslinked products such as Sepracoat were shown to be prophylactically effective but remain ineffective or lack data supporting the reduction of IUAs when applied after tissue injury has occurred (17). Viscoelastic forms have shown promising clinical results in the overall reduction of IUAs but remain subject to premature dilution and are challenged to stretch overall persistence times. To date, there is still no single modality proven to be satisfactorily effective in preventing post-operative adhesion formation for hysteroscopic use (18).

In-situ forming hydrogels offer several advantages in use as an adhesion barrier. The liquid nature of the precursors allows for ease-of-use, minimized invasiveness and thorough application to the entire uterine cavity. After formation of the gel through crosslinking, the barrier is more resistant to expulsion from the uterine cavity and to premature dilution. Hydrogel formulations are generally described that can achieve persistence times designed to the prevention of IUAs. Previous efforts for the application of hydrogels for the prevention of intrauterine adhesions is described in published U.S. patent application 2005/0266086 to Sawhney (hereinafter the '086 application), entitled “Intrauterine Applications of Materials Formed In Situ,” incorporated herein by reference. The Example used a material referred to as SPRAYGEL, which was developed and demonstrated to be useful for prevention intraperitoneal adhesion formation (5,6,7), see Mettler et al., “Prospective Clinical Trial of SprayGel as a Barrier to Adhesion Formation: An Interim Analysis,” Journal of the American Association of Gynecological Laparoscopists, (August 2003) 10 (3), 339-344, incorporated herein by reference. SPRAYGEL was comprised of two liquids (one clear and one blue) that each contain chemically distinct polymeric precursors which, when mixed together, rapidly cross-link to form a biocompatible absorbable hydrogel in situ. Additional details for SPRAYGEL are provided in U.S. Pat. No. 7,009,034 to Pathak et al., entitled “Biocompatible Crosslinked Polymers,” incorporated herein by reference herein. The concept of a hydrogel material for prevention of intrauterine adhesions is known, but with limited success, as evaluations were conducted using compositions and devices designed for intraperitoneal applications (19, 20). The intrauterine environment presents unique challenges of restricted space, contractions of the uterine muscle, and exit pathway out of the body, different healing mechanism after injury, and other differences, relative to the intraperitoneal environment. Thus, specific compositions and delivery devices are desirable to achieve target results for intrauterine adhesion prevention.

### Hydrogels for Medical Applications

Hydrogels generally are considered insoluble materials that absorb water with swelling to form elastic three-dimensional networks. See, e.g., Park, et al., Biodegradable Hydrogels for Drug Delivery, Technomic Pub. Co., Lancaster, Pa. (1993). Covalently crosslinked networks of hydrophilic polymers are traditionally denoted as hydrogels in the hydrated state. Precursors of the hydrogels are generally water soluble polymers that become insoluble upon suitable crosslinking. As noted in the following, hydrogels are known based on various chemistries using suitable hydrophilic polymers. In some contexts, swelling can refer to continued volume or weight changes following initial formation of a crosslinked insoluble structure, in which case the specification of timing is appropriate. While a transition from a dried state to a hydrated state would results in weight increases and generally some volume increase, changes from an initial state formed in aqueous solution to an aged state may or may not involve an increase in weight or volume over time, and may results in some time windows in decreases.

Crosslinkable solutions for use in the methods described herein include precursor solutions that may be used to form hydrogel structures in situ in lumens or voids within the patient, and that form physical crosslinks, chemical crosslinks, or both. Physical crosslinks may result from complexation, hydrogen bonding, physical entanglement n, Van der Waals interactions, ionic bonding, and other interactions, and may be initiated by radiation delivered to the site, by mixing two components that are physically separated until combined in situ, or as a consequence of a prevalent condition in the physiological environment, such as temperature, pH, ionic strength, other environmental conditions or a combination thereof. Chemical crosslinking may be accomplished by any of a number of mechanisms, including free radical polymerization, condensation polymerization, anionic or cationic polymerization, step growth polymerization, or other classes of chemical reactions. Where two solutions are employed, each solution can comprise one component of a co-initiating system and crosslink on mixing. The solutions can be separately stored and mixed when delivered into a tissue lumen. Suitable applicators are described in detail herein for precursors based on one, two or more precursor solutions.

Hydrogels may be crosslinked spontaneously from at least one precursor without requiring the use of a separate energy source. Such systems allow for control of the crosslinking process, e.g., because a large viscosity increase of materials flowing through a delivery device does not occur until after the precursors contact the environment outside of the applicator. In the case of a dual-component system, mixing of the two solutions takes place so that the solutions are fluid while passing through the device. If desired, one or both crosslinkable precursor solutions may contain contrast agents or other means for visualizing the hydrogel implant. The crosslinkable solutions may contain a bioactive drug or other therapeutic compound that is entrapped in the resulting implant, so that the hydrogel implant serves a drug delivery function with gradual drug elution.

Additional properties of the hydrogel system can be selected according to the intended application. For example, if the hydrogel implant is to be used to temporarily occlude a reproductive organ, such as the uterine cavity, it can be desirable that the hydrogel system undergo moderate swelling to conform to irregular geometries and be biodegradable within the timeframe of a single menstrual cycle. The hydrogel is preferably soft and of a modulus or stiffness that is lower than that of uterine tissue in a non-gravid uterus. More generally, the materials should be selected on the basis of exhibited biocompatibility and lack of toxicity.

Additionally, the hydrogel system solutions maybe prepared without harmful or toxic solvents. In general, the solutions are substantially soluble in water to allow application in a physiologically-compatible solution, such as buffered isotonic saline. The hydrogels may be biodegradable, so that the hydrogel implant does not have to be retrieved from the body. Biodegradability, as used herein, refers to the predictable disintegration of the hydrogel into molecules small enough to be metabolized, purged or excreted under normal physiological conditions. Biodegradability may occur by, e.g., hydrolysis, enzymatic action, reversal of physical crosslinking by an instilled agent, or cell-mediated destruction.

### Monomers and Macromers for Chemical Crosslinking

Monomers capable of being crosslinked to form a biocompatible implant may be used. The monomers may be small molecules, such as acrylic acid or vinyl caprolactam, larger molecules containing polymerizable groups, such as acrylate-capped polyethylene glycol (PEG-diacrylate), or other polymers containing ethylenically-unsaturated groups, such as those of U.S. Pat. No. 4,826,945 to Cohn et al., entitled “Biodegradable Polymeric Materials Based on Polyether Glycols, Processes for Preparation Thereof and Surgical Articles made Therefrom,” U.S. Pat. No. 5,160,745, to De Luca et al. entitled “Biodegradable Microspheres as a Carrier for Macromolecules,”, or U.S. Pat. No. 5,410,016 to Hubbell et al. (hereinafter the '016 patent), entitled “Photopolymerizable, Biodegradable Hydrogels as Tissue Contacting Materials and Controlled-Release Carriers,” all of which are incorporated herein by reference.

Water soluble polymerizable polymeric monomers having overall a functionality >2 (i.e., that form crosslinked networks on polymerization) and that form hydrogels may be referred to herein as macromers.

Several functional groups may be used to facilitate chemical crosslinking reactions. When these functional groups are self polymerizing, such as ethylenically unsaturated functional groups, the macromer alone is sufficient to result in the formation of a hydrogel, when polymerization is initiated with appropriate agents. Where two solutions are employed, each solution preferably contains one component of a co-initiating system and crosslink on contact. The solutions are stored in separate compartments of a delivery system, and mix either when deposited on or within the tissue.

An example of an initiating system suitable for use in forming the hydrogels is the combination of a peroxygen compound in one solution, and a reactive ion, such as a transition metal, in another. Other initiating systems such as pH, thermally or photochemically initiated systems may also be used. Other means for crosslinking macromers to form hydrogel implants in situ also may be advantageously used, including macromers that contain groups that demonstrate activity towards functional groups such as amines, imines, thiols, carboxyls, isocyanates, urethanes, amides, thiocyanates, hydroxyls, or the like, which may be naturally present in, on, or around tissue. Alternatively, such functional groups may be provided in a second compositional component, which can be a small molecule or a second macromer, as part of the hydrogel system.

Suitable hydrogel systems are those biocompatible single component or multi-component systems that spontaneously crosslink when the components are activated by an initiating system, by a change in environment, or by mixing two components, although if two or more components are used, they may be individually stable. Such systems include, for example, contain macromers that are di or multifunctional amines in one component and di or multifunctional oxirane containing moieties in the other component. Other initiator systems, such as components of redox type initiators, also may be used. The mixing of the two or more solutions may result in either an addition or condensation polymerization that further leads to the formation of an implant. Free radical driven crosslinking systems that depend on thermal initiation or photoinitiation may also be used to trigger the polymerization of ethylenically unsaturated monomers or macromers to form hydrogels.

Monomers may include the biodegradable, water-soluble macromers described in the '016 patent. These monomers are characterized by having at least two polymerizable groups, separated by at least one degradable region. When polymerized in water, they form coherent gels that persist until eliminated by self-degradation. In one embodiment, the macromer is formed with a core of a polymer that is water soluble and biocompatible, such as the polyalkylene oxide polyethylene glycol, flanked by hydroxy acids such as lactic acid, having acrylate groups coupled thereto. In general, monomers, in addition to being biodegradable, biocompatible, and non-toxic, also can be at least somewhat elastic after crosslinking or curing.

It has been determined that for certain crosslinked polymers. use of monomers with longer distances between crosslinks generally form hydrogels that are softer, more compliant, and more elastic. Thus, in the polymers such as those of the '016 patent, increased length of the water-soluble segment, such as polyethylene glycol, tends to enhance elasticity. Molecular weights in the range of 10,000 to 35,000 grams/mole (g/mol) of polyethylene glycol provide particularly useful properties for such applications, although molecular weight ranges from 1,000 to 500,000 g/mol can also be useful.

### Crosslinking Reactions and Initiating Systems

Crosslinking reactions can take place due to nucleophilic-electrophilic substitutions, free radical reactions, oxidation/reduction reactions or the like. These reactions can be initiated, by mixing, heat, pH change, radiation, and/or pressure. In one component systems described herein, body heat can be used as an initiator, or pH change associated with tissue contact, but radiation can be effective for more rapid crosslinking. For two component systems described herein, mixing can be used to initiate well-controlled hydrogel delivery, although other initiators can be used.

Metal ions may be used either as an oxidizer or as a reductant in redox initiating systems. For example, ferrous ions may be used in combination with a peroxide or hydroperoxide to initiate polymerization, or as parts of a polymerization system. In this case, the ferrous ions serve as a reductant. In other previously known initiating systems, metal ions serve as an oxidant. For example, the ceric ion (4+ valence state of cerium) interacts with various organic groups, including carboxylic acids and urethanes, to remove an electron to the metal ion, and leave an initiating radical behind on the organic group. In such a system, the metal ion acts as an oxidizer.

Thermal initiating systems may be used rather than the redox-type systems described hereinabove. Several commercially available low temperature free radical initiators, such as V-044, available from Wako Chemicals USA, Inc., Richmond, Va., may be used to initiate free radical crosslinking reactions at body temperatures to form hydrogel implants with the aforementioned monomers. Initiators such as potassium and sodium persulfate, various peroxy and hydroperoxyl compounds can be used. Photopolymerization initiation systems containing UV initiators such as Irgacure 651 (Ciba Geigy) can also be used.

For the applications described herein, the crosslinking reactions generally are designed to occur in aqueous solution under physiological conditions. Thus, the crosslinking reactions occur “in situ”, meaning they occur at local sites such as on organs or tissues in a living animal or human body. Due to the in situ nature of the reaction, the crosslinking reactions can be designed not to release undesirable amounts of heat of polymerization. Crosslinking times for desirable procedures can be set accordingly. Certain functional groups, such as alcohols or carboxylic acids, do not normally react with other functional groups, such as amines, under physiological pH (e.g., pH 7.2-11.0, 37° C.). However, such functional groups can be made more reactive by using an activating group such as N-hydroxysuccinimide. Several methods for activating such functional groups are known in the art. Suitable activating groups include for example, carbonyldiimidazole, sulfonyl chloride, aryl halides, sulfosuccinimidyl esters, N-hydroxysuccinimidyl ester, succinimidyl ester, epoxide, aldehyde, maleimides, imidoesters and the like. The N-hydroxysuccinimide esters or N-hydroxysulfosuccinimide groups are particularly desirable for crosslinking of proteins or amine functionalized polymers such as amino terminated polyethylene glycol (“APEG”).

Aqueous solutions of NHS based crosslinkers and functional polymers preferably are made just before the crosslinking reaction due to reaction of NHS groups with water. Longer “pot life” may be obtained by keeping these solutions at lower pH (pH 4-5).

The crosslinking density of the resultant biocompatible crosslinked polymer is controlled by the overall molecular weight of the crosslinker and functional polymer and the number of functional groups available per molecule. A lower molecular weight between crosslinks such as 600 Da can give much higher crosslinking density as compared to a higher molecular weight such as 10,000 Da. Higher molecular weight functional polymers generally can be used to obtain more elastic gels.

The crosslinking density also may be controlled by the overall percent solids of the crosslinker and functional polymer solutions. Increasing the percent solids increases the probability that an electrophilic functional group will combine with a nucleophilic functional group prior to inactivation by hydrolysis. Yet another method to control crosslink density is by adjusting the stoichiometry of nucleophilic functional groups to electrophilic functional groups. A one to one ratio leads to the highest crosslink density.

Hydro Gels with Nucleophilic/Electrophilic Crosslinking

Hydrogels that are particularly suitable for the applications described herein can be delivered through less invasive means, such as a catheter with a small diameter. Thus, the hydrogel itself may be either thixotropic or may be fully formed in-situ after delivery. The hydrogels of particular interest generally begin as precursors that can react to form into a gel upon crosslinking by nucleophilic substitution. In some embodiments, the crosslinking reaction occurs slowly under neutral conditions, but the addition of an accelerator, such as a basic buffer, accelerates the reaction. For the hydrogels of particular interest herein, suitable buffers are near a neutral pH, and include, for example, include borate, phosphate, citrate, bicarbonate, CHES, TAPS, bicine, Tris, tricine, or the like. The selected hydrogel precursors can be initially mixed to have a pH different from neutral to provide for slow crosslinking until mixed with the accelerant buffer. Other methods of triggering the polymerization reaction, such as heat, light etc. can also be advantageously used, if appropriate polymerization systems and precursors are selected. For example, polyethylene glycol di or mutiacrylate polymers can be used to form the hydrogel with a single precursor, which can be polymerized using thermal initiators of free radical polymerization or photo initiators of free radical polymerization. In general, two component based systems are desirable, since they do not rely on any external energy source and can enable rapid crosslinking without concerns for shadowing or heat requirement or generation. The conditions can be controlled to obtain crosslinking and gel formation suitable for the delivery process using the applicator described above in the context of the delivery system. Generally, the crosslinking begins in the catheter of the delivery system, but does not complete sufficiently to limit flow from the catheter into the patient. The hydrogel can set sufficiently crosslinked to remain in place in a reasonable period of time and can fully crosslink following completion of the delivery procedure.

It is sometimes useful to provide color by adding a colored visualization agent to hydrogel precursors before crosslinking. The visualization agent may serve to help a used visualize the disposition of the hydrogel. For example, when filling a uterus, a visualization agent will help to distinguish the hydrogel from other fluids. Further, the hue of a colored hydrogel may provide information about the concentration of the precursors in the hydrogel or the degree of mixing of physiological fluids into the hydrogel. A dark color hydrogel may indicate a concentration of precursors that is high relative to a lighter hued hydrogel made from the same precursor solutions. The coloring agent may be present in a premixed amount that is already selected for the application. Colors such as blue and green provide an appropriate contrast to blood. An embodiment of the hydrogel uses biocompatible crosslinked polymers formed from the reaction of precursors having electrophilic functional group and nucleophilic functional groups. The precursors are generally water soluble, non-toxic, and biologically acceptable.

A precursor may be multifunctional, to enhance the rate of polymerization. Depending on the polymerization chemistry and the end groups selected, the precursors may be self reacting (e.g., with acrylate and methacrylate based systems), or may have complementary end groups that react with each other. For example, in an electrophilic-nucleophilic reaction system, a precursor comprises two or more electrophilic or nucleophilic functional groups, such that a nucleophilic functional group on one precursor may react with an electrophilic functional group on another precursor to form a covalent bond. If a precursor has more than two functional groups, the precursor molecule can participate in crosslinking reactions, and generally the hydrogels are relatively highly crosslinked.

A hydrogel for use on a patient's tissue can comprise water, a biocompatible visualization agent, and crosslinked hydrophilic polymers that form a hydrogel after delivery within the uterine cavity. The visualization agent can reflect or emit light at a wavelength detectable to a human eye so that a user applying the hydrogel can observe the gel and also estimate its bulk.

The hydrogels for intrauterine placement can have moderate swelling with sufficient swelling to facilitate filling the space but not excessive swelling to result in uncomfortable pressure on the patient. In some embodiments, the hydrogel can have a swelling of no more than 300 weight percent, in further embodiments from about 10 wt % to about 200 wt % and in further embodiments form about 20 wt % to about 100 wt %. In alternative embodiments, the hydrogel can experience syneresis, or a shrinking on a weight basis, generally also on a volume basis, following initial formation, which for convenience is referred to as a negative swelling. Thus, overall swelling can be from about −25 wt % to about 300 wt %, in further embodiments from about −15 wt % to 200 wt %, and in other embodiments from about −10 wt % to about 100 wt %. Swelling (positive or negative) can be determined by the weight of polymer with aqueous solution of buffered saline absorbed into the polymer after 24 hours of contact with the aqueous environment relative to the weight of the polymer and absorbed aqueous solution following crosslinking into an insoluble mass, which generally occurs after several seconds. The hydrogel can be biodegradable so that the uterine space clears after a suitable period of time that the healing process does not trade the hydrogel material itself. In some embodiments, the hydrogel is fully biodegraded in from about 3 hours to about 21 days, in further embodiments from about 3 days to about 14 days, and in additional embodiments from about 5 days to about 8 days. For certain applications, such as drug delivery, it may be desirable for the hydrogel to biodegrade over a longer time period, for example, a 30 days or longer. Also, the hydrogel can be selected to be soft so as to be gentle on the tissue, yet not so soft as to be extrudable from the uterus, resulting in unpredictable persistence within the cavity. Specifically, the hydrogel can have a Young's (elastic) modulus from about 1 kPa to about 300 kPa, in further embodiments from about 5 kPa to about 250 kPa and in additional embodiments from about 5 kPa to about 200 kPa. A person of ordinary skill in the art will recognize that additional ranges of swelling, degradation rate and Young's modulus within the explicit ranges above are contemplated and are within the present disclosure.

Natural polymers, for example proteins or glycosaminoglycans, e.g., collagen, fibrinogen, albumin, and fibrin, may be crosslinked using reactive precursor species with electrophilic functional groups. Natural polymers are proteolytically degraded by proteases present in the body. The precursors may have biologically inert and water soluble cores. When the core is a polymeric region that is water soluble, suitable polymers that may be used include: polyether, for example, polyalkylene oxides such as polyethylene glycol(“PEG”), polyethylene oxide (“PEO”), polyethylene oxide-co-polypropylene oxide (“PPO”), co-polyethylene oxide block or random copolymers, and polyvinyl alcohol (“PVA”); poly (vinyl pyrrolidinone) (“PVP”); poly (amino acids); dextran and proteins such as albumin. The polyethers and more particularly poly(oxyalkylenes) or poly(ethylene glycol) or polyethylene glycol can provide desirable properties for the hydrogels.

Synthetic polymers and reactive precursor species may have electrophilic functional groups that are, for example, carbodiimidazole, sulfonyl chloride, chlorocarbonates, n-hydroxysuccinimidyl ester, succinimidyl ester or sulfasuccinimidyl esters. In some embodiments of particular interest, the electrophylic functional groups comprise N-hydroxy succinimidyl succinate (SS) esters that provide desirable crosslinking rates to form the hydrogel and degradation rates for the hydrogel subsequently in vivo.formed hydrogel. The term synthetic means a molecule that is not found in nature, e.g., polyethylene glycol. The nucleophilic functional groups may be, for example, amine, such as primary amines, hydroxyl, carboxyl, and thiol. Primary amines can be desirable reactants with NHS electrophilic groups. The polymers in embodiments of particular interest have a polyalkylene glycol portion and can be polyethylene glycol based. The polyethylene glycol-based polymer precursors can have a branched core to provide a selected number of arms that provide a plurality of crosslinking functional groups. The polymers generally also have a hydrolytically biodegradable portion or linkage, for example an ester, carbonate, or an enzymatically degradable amide linkage. Several such linkages are well known in the art and originate from alpha-hydroxy acids, their cyclic dimers, or other chemical species used to synthesize biodegradable articles, such as, glycolide, dl-lactide, l-lactide, caprolactone, dioxanone, trimethylene carbonate or a copolymer thereof. In some embodiments, a reactive precursor species can have two to ten nucleophilic functional groups each, and corresponding reactive precursor species can have two to ten electrophilic functional groups each.

In some embodiments, a mixture or a process of mixing hydrophilic reactive precursor species involves having nucleophilic functional groups with hydrophilic reactive precursor species and having electrophilic functional groups such that they form a mixture that crosslinks. If the mixture reacts relatively slowly under neutral conditions, the precursors can be mixed and placed into a syringe or comparable reservoir of the delivery system shortly before administration. An accelerant can be placed into the other syringe or comparable fluid reservoir. The accelerant can be mixed with the precursor blend during delivery to initiate more rapid crosslinking due to a pH change or other suitable property of the blend. Since the precursors can be well mixed prior to administration, the mixing process can be more complete in the delivery system such that well mixed compositions are delivered into the catheter for intrauterine delivery. Hydrophilic reactive precursor species can be dissolved in buffered water such that they provide low viscosity solutions that readily mix and flow when contacting the tissue and are effective in completely filling and casting out the interior of the uterine cavity. The use of a small molecule crosslinker for one precursor provides for a relatively low viscosity of the blended precursors prior to extensive crosslinking such that the blended hydrogel precursors can be delivered through a thin catheter while crosslinking initiates and the fluid conforms to the shape of the uterine cavity, but then relatively rapid crosslinking provides for stabilization of the hydrogel within the uterus in a reasonable period of time.

As the precursor blend flows across the tissue, the hydrogel forming during the crosslinking process conforms to the shape of the small features of the tissue such as bumps, crevices and any deviation from surface smoothness, although perfect conformation is not necessary. Without limiting to a particular theory of operation, it is believed that reactive precursor species that crosslink appropriately quickly after contacting a tissue surface form a three-dimensional structure filling the space into which they are delivered. This three dimensional structure also is resistant to expulsion from the uterine cavity, thus serving to keep the uterine walls apart and prevent the formation of scar bridges, or adhesions. Over time, the hydrogels degrade and naturally exit the uterine cavity by either systemic absorption, or mostly as discharge through the cervix and vagina.

Suitable crosslinking times vary for different applications. In most applications, the crosslinking reaction leading to gelation occurs within about 5 minutes, in some embodiments, within about 1 minutes, and in further embodiments from about 2 second to about 30 seconds from initiation of delivery to gelation. A person of ordinary skill in the art will recognize that additional ranges of gelation time within the explicit ranges above are contemplated and are within the present disclosure. These gelation times do not necessarily correspond with full crosslinking which can occur over a longer time period, but the gelation times correspond with reaching a point of crosslinking in which the hydrogel no longer is flowable. Crosslinking times for in situ systems are a combination of several factors, including relative concentrations of reactive precursors, the molar ratios of the reactive ends, temperature, and resulting pH after mixing. Gel times may be varied by one or more of altering the pH, temperature, or buffer salt strength of the “accelerator” portion of the in situ system, if present.

### Biodegradable Linkages

If it is desired that the biocompatible crosslinked polymer be biodegradable or absorbable, one or more precursors having biodegradable linkages may be used. The biodegradable linkage optionally also may serve as the water soluble core of one or more of the precursors. In the alternative, or in addition, the functional groups of the precursors may be chosen such that the product of the reaction between them results in a biodegradable linkage. For each approach, biodegradable linkages may be chosen such that the resulting biodegradable biocompatible crosslinked polymer degrades or is absorbed in a desired period of time. Generally, biodegradable linkages are selected that degrade under physiological conditions into non-toxic products.

The biodegradable linkage may be chemically or enzymatically hydrolyzable or absorbable. Illustrative enzymatically hydrolyzable biodegradable linkages include peptidic linkages cleavable by metalloproteinases and collagenases. Additional illustrative biodegradable linkages include polymers and copolymers of poly(hydroxy acid)s, poly(orthocarbonate)s, poly(anhydride)s, poly(lactone)s, poly(aminoacid)s, poly(carbonate)s, and poly(phosphonate)s.

### Visualization Agents

Where convenient, the biocompatible crosslinked hydrogel polymer may contain visualization agents to improve their visibility during surgical procedures. Visualization agents are especially useful when used in MIS procedures, due among other reasons to their improved visibility on a color monitor.

Visualization agents may be selected from among any of the various non-toxic colored substances suitable for use in medical implantable medical devices, such as FD&C BLUE dyes 1, 2, 3 and 6, indocyanine green, or colored dyes normally found in synthetic surgical sutures. In some embodiments, green or blue colors are desirable because these have better visibility in presence of blood or on a pink or white tissue background.

The visualization agent may be present with one or more precursors for delivery or with an accelerator. The selected colored substance may or may not become chemically bound to the hydrogel. Additional visualization agents may be used, such as fluorescent (e.g., green or yellow fluorescent under visible light) compounds (e.g., fluorescein or eosin), x-ray contrast agents (e.g., iodinated compounds) for visibility under x-ray imaging equipment, ultrasonic contrast agents, or MRI contrast agents (e.g., Gadolinium containing compounds). Visualization agents may also be biologically active agents suspended or dissolved within the hydrogel matrix, or the materials used to encapsulate the biologically active agents.

As noted above, visually observable visualization agents can be advantageously used for some embodiments. Wavelengths of light from about 400 to 750 nm are observable to the human as colors (R. K. Hobbie, Intermediate Physics for Medicine and Biology, 2^{nd} Ed., pages 371-373). The user may use visualization agents to see the hydrogel with the human eye or with the aid of an imaging device that detects visually observable visualization agents, e.g., a videocamera that is used during operative hysteroscopy. A visually observable visualization agent is an agent that has a color detectable by a human eye. A characteristic of providing imaging to an X-ray or MRI machine is not a characteristic sufficient to establish function as a visually observable visualization agent. An alternative embodiment is a visualization agent that may not normally be seem by the human eye but is detectable at a different wavelength, e.g., the infra red or ultraviolet, when used in combination with a suitable imaging device, e.g., an appropriately equipped videocamera.

In some embodiments, the visualization agent is present in the hydrogel system during application into a void such as the uterus through the delivery system described herein. In such applications, the target tissue of the intrauterine surface is not, or cannot be, visualized. The presence of the visualization agent in application may enable the user to detect when the cavity has been sufficiently filled with material through the presence of excess exiting the target cavity. In the case of an intrauterine application following a surgical intervention, the presence of a blue or green visualization aid allows for differentiation from excess bodily blood and fluids resultant of the surgery, as well as confirmation that the application and hydrogel crosslinking has occurred.

Suitable biocompatible visualization agents are FD&C BLUE #1, FD&C BLUE #2 Indocyanine green. Methylene blue, while providing appropriate potential for visualization, is less preferred due to reports of allergenic potential in gynecological procedures, or other medically acceptable colorants and dyes that provide a contrasting color with red serosanguinous fluids. One or both of these agents can be present in the final electrophilic-nucleophilic reactive precursor species mix at a concentration of more than 0.05 mg/ml and in some embodiments in a concentration range of at least 0.1 to about 12 mg/ml, and in further embodiments in the range of 0.1 to 4.0 mg/ml, although greater concentrations may potentially be used, up to the limit of solubility of the visualization agent. These concentration ranges were found to give a color to the hydrogel that was desirable without interfering with crosslinking times (as measured by the time for the reactive precursor species to gel) and were determined to be more radiation stable than other visualization agents such as methylene blue. The visualization agent may also be a fluorescent molecule. The visualization agent is generally not covalently linked to the hydrogel. A person of ordinary skill in the art will recognize that additional ranges of visualization agent concentrations within the explicit ranges above are contemplated and are within the present disclosure.

In some embodiments, a hydrogel is selected and delivered that at least partially fills a uterus, and in embodiments of particular interest, the hydrogel substantially fills a uterus. Thus, upon fully crosslinking, the hydrogel is shaped like an interior of a uterus. While filling the uterus, a hydrogel can form a coating on at least a portion of an intrauterine tissue. In some embodiments, a hydrogel substantially fills a uterus and has contact with substantially all of the tissues exposed inside the uterus and in the cervical canal. The introduction of fluent precursor(s) or precursor solutions into a uterus that form a hydrogel having a volume that is essentially equal to the volume of the fluent precursor(s) or precursor solutions, with some potential adjustment based on swelling, can contact substantially all of the tissues exposed inside the uterus because a fluid will conform to the shape of the tissues. Nonetheless, it is appreciated by persons of ordinary skill in the art that even substantially complete contact may suffer from imperfections.

In some embodiments, a method is used to form a hydrogel on a tissue until the color of the hydrogel indicates that a predetermined volume of hydrogel has been deposited on the tissue or within the space. The precursors are continually introduced into the space until the color of the materials that enter that space and flow out are deemed to have achieved a suitable content, as indicated by observation of the visualization agent disposed in the materials that flow out. For example, two fluent precursors associated with a blue dye are introduced into a uterus and pumped therein until the color of materials exiting the uterus indicates that unwanted fluids have been washed out of the uterus and the uterus is substantially full of the precursors.

### Catheter Systems For Forming Hydrogel Implants In Situ

The catheter systems taught herein provide for desired functionality for delivery of a polymer or other fluid as well as removal of fluid for corresponding applications. In particular, the catheter systems provide for desirable placement of a fluid such as hydrogels, in the uterus and maintenance of at appropriate locations to inhibit adhesion formation, although the catheter systems are suitable for other purposes involving movement of fluids into or out from the intrauterine cavity. For an ordinary practitioner in the art, the catheter system provides a graspable structure that allows for placement and actuation with a single hand to leave another hand free for other functions, although a practitioner with physical challenges can adapt the catheter system appropriately for their needs. In some embodiments, the catheter system used as an applicator incorporates a design in which compositions from separate syringes are more actively mixed and then directed to a narrow tube or catheter. The improved catheter system designs can comprise a cervical cap secured to a tubular element with a size suitable for placement on the catheter extending in a proximal direction (toward the practitioner) such that the cervical cap can be positioned against the cervix at a position to have the catheter extending into the patient's uterus a desired distance. The cervical cap can be left in place when the catheter is removed to avoid disturbance of the hydrogel when the catheter is removed, and the cervical cap than subsequently be removed to leave filler within the cervix to block adhesion formation, which can be particularly problematic near the internal OS. In additional or alternative embodiments, a cervical plug can be used to provide additional stabilization of the hydrogel within the uterus.

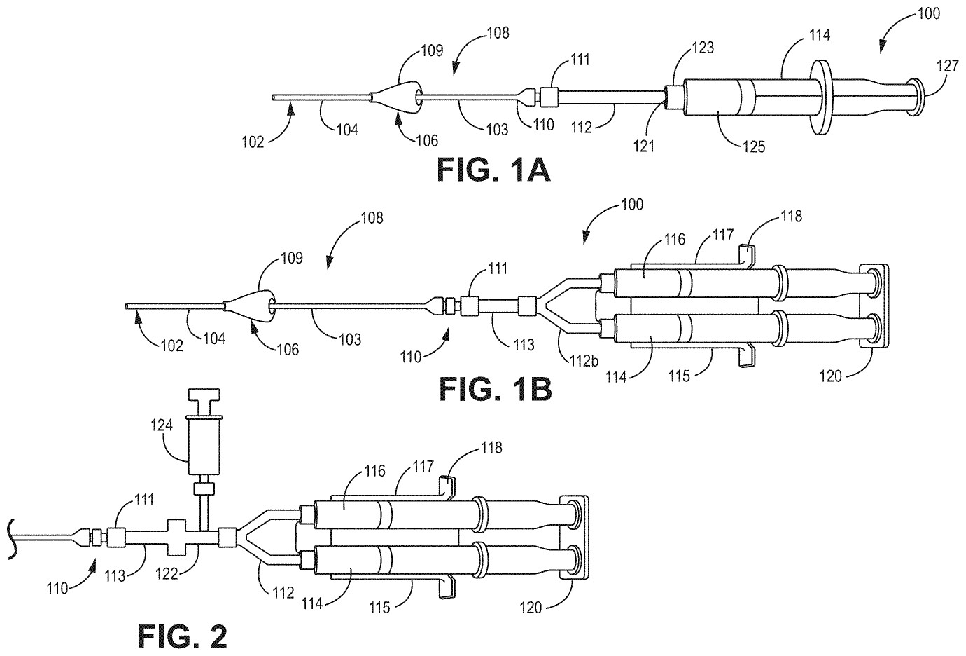

With respect to illustrative transcervical access systems, FIG. 1A and FIG. 1B respectively display an applicator suitable for a single component hydrogel precursor or a two component hydrogel precursor. If more than two components are delivered, such as three, four or more components, the figures can be generalized by a person of ordinary skill in the art based on these teachings. Two component systems are exemplified and are of particular interest for commercial products, so most of the following discussion focuses on the two component applicator of FIG. 1B, but the discussion readily generalizes for the other embodiments. Similarly, other fluids can be delivered similarly to the hydrogel precursors. Also, with an empty syringe compartment, the catheter system can be used to extract out fluid, which can be any composition that can be pulled into the catheter.

Referring to FIG. 1A, an illustrative transcervical access system constructed in accordance with the principles of the protocols herein is described. Transcervical access system 100 comprises single-lumen catheter 108 having proximal end 110 and distal end 102. Distal end 102 generally comprises one or more delivery ports, as described in detail below. Proximal end 110 is attached to an extender element 112 through a fitting 111, such as a standard luer lock fitting with male and female elements respectively on catheter 108 with a single lumen and extender element 112. In general, transcervical access system 100 has an egress limiter 106 along catheter 108. Egress limiter 106 sets a depth for insertion of the catheter into the patient and stops flow out from the cervix during dispensing of the hydrogel into the uterus. In some embodiments, egress limiter 106 is adjustable, such as being slidable along catheter 108 to adjust the length of catheter segment 104, distal to the adjustable egress limiter 106. Thus, the practitioner can adjust egress limiter 106 to a desired depth for insertion into the patient to help ensure uniform delivery of the hydrogel, as described further below. In embodiments of particular interest, egress limiter 106 comprises a support sheath 103 and cap element 109, as described in detail below, in which support sheath 103 can provide a stiffening function as well as facilitating handling of cap element 109.

As shown in FIG. 1A, the distal length of catheter 108 from the distal end of installation tip 102 to the proximal end of cap element 109 may be from about 5 cm to about 15 cm. In some embodiments, the distal length may be from about 7 cm to about 10 cm. Also shown in FIG. 1A, the proximal length of the catheter from the proximal end of cap element 109 to the proximal end of fitting 111 may be approximately 4 cm to about 20 cm. In some embodiments, the proximal length may be from about 7 cm to about 9 cm. One of ordinary skill in the art will appreciate that the selected length of the catheter and the position of the reference marker will depend on various factors, such as the patient's anatomy, the application conditions, and the practitioner's preferences and that additional ranges of values within the explicit ranges above are contemplated and are within the present disclosure. For example, a practitioner may choose a longer catheter for a transcervical assisted laparoscopic procedure than for a transvaginal intrauterine procedure and this preference could be accommodated by using a longer catheter length while independently maintaining a medically appropriate length of catheter segment 104, which can be adapted from the more specific teachings for a cervical approach by a person of ordinary skill in the art. For commercial devices, generally various catheter lengths can be available for selection by the health care provider that can be connected to fitting 111, although in some embodiments described below, the length is adjustable such that the same components can be used to provide different lengths from the closure to the distal end. Cap element 109 may have a conical shape, as shown, or other shape to act as a backstop or egress limiter for the prevention of excess material run off during the hydrogel application, for providing gently pressurize tamponade-like fill of the hydrogel, as well as to guide the health care professional for placement at the selected position.

Extender element 112 generally has a larger diameter than catheter 108 and is stiffer. Extender element 112 can provide for convenient manipulation of catheter 108 for insertion into the patient and provide the practitioner better positioning for the procedure while limiting the length of catheter 108, which may be more difficult to manipulate if it is too long. Extender element 112 has a connector 121 at its proximal end, such as a luer connector, which connects to syringe 114 at a connecter 123 on the syringe. Syringe 114 can be a conventional syringe loaded with hydrogel precursor in reservoir 125 and any other additives, such as one or more additives described above. Hydrogel precursor can be delivered through pressing of plunger 127 to deliver hydrogel precursor through extender element 112 into catheter 108 into the patient from distal end 102.

An alternative embodiment for the simultaneous delivery of two fluids is shown in FIG. 1B. Catheter 108 and egress limiter 106 can be the same as in FIG. 1A. Catheter 108 connects to a Y-connector 112b at fitting 111, such as a luer fitting or the like. Y-connector 112b can comprise a static mixing element, such as within tubing segment 113. Alternatively, static mixing element can be substituted with simply a tube in which mixing occurs more gradually. In any case, mixing of a first solution 114 and a second solution 116 occurs or begins within Y connector 112b. Y-connector 112b can comprise the mixing element in its outflow channel, or a separate section of tubing connected to the Y-structure, and the static mixing element can comprise flow-altering baffles, such as a helical, plate-type or other flow diverter known in the art to induce turbulence within a tubular channel, to encourage appropriate mixing of the solutions. Y-connector 112b has a fitting 111, such as a Luer connector for attachment of catheter 108, and connections to syringes 115, 117, which may or may not be releasable. First solution 114 can be a first precursor or a mixture of a first precursor and a second precursor if the reaction does not occur to an undesirable amount during relevant time scales without an accelerator or catalyst, and respectively, the second solution can be a second precursor or an accelerator/catalyst. Syringes 115 and 117 generally are held via a molded syringe holder 118 or the like to provide for convenient handling by the health care professional during use. An optional fixed ratio of solution delivery ratio can be maintained by an optional plunger cap 120. If the internal diameters of syringes 115, 117 are the same, movement of plunger cap 120 would deliver a 1:1 ratio of volumes, but the internal diameters can be selected to provide a different volume ratio if desired. The outer diameters of syringes 115, 117 may or may not track the inner diameters depending on the syringe wall thicknesses.

An alternative embodiment of transcervical access system 100 is depicted in FIG. 2. In this embodiment, a T-branch fitting 122 is located within Y-connector 112 proximal to tubing segment 113. In some embodiments, T-branch fitting 122 can be secured with connectors, such as Luer connectors on the respective ends of the three branches, although in some embodiments, T-branch connector 122 can be molded to be integral with one or more of adjacent components. Similarly, another Y-branch or other fitting design can be used on place of T-branch fitting T-branch fitting 122 can be connected to a syringe 124 or other fluid source, which can provide an inert flush, such as buffered saline, to help clear the catheter or for the delivery of a therapeutic or other desirable liquid.

Transcervical access system 100 and components thereof may be fabricated of any of a wide variety of materials that are sufficiently flexible and biocompatible, and different components can be assembled from appropriate materials for that component. Some components can be readily adapted from commercially available parts. For example, silicone rubber, natural rubber, polyisoprene, butyl rubber, polyethylene, polypropylene, nylon, polyvinylchloride, polyether block amide, polyesters (such as polyethylene terephthalate-PET), polycarbonates, polyurethanes, polyolefins, polysiloxanes, copolymers thereof, mixtures thereof and other similar materials are suitable. In some embodiments, the delivery system comprises a soft installation tip material to reduce traumatic injury to the uterine surface during insertion and injection of the mixed fluid, and materials for the installation tip are described further below.

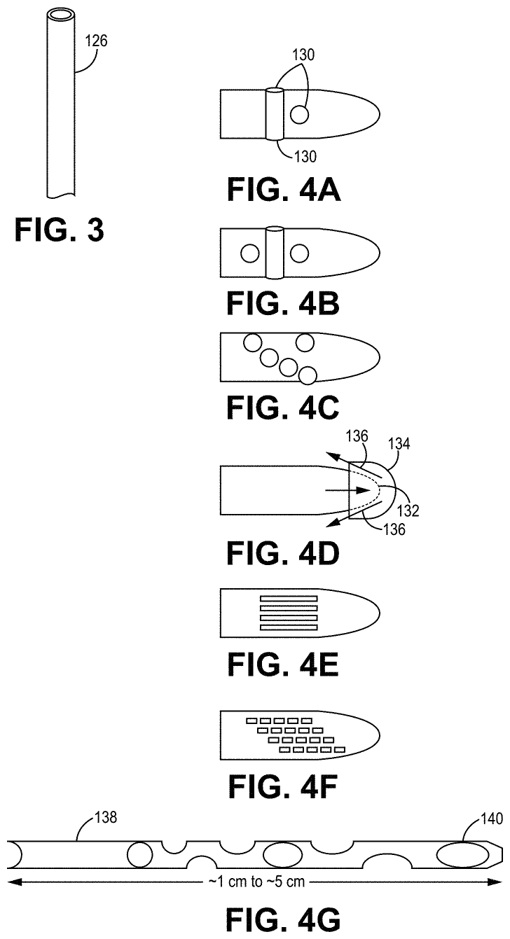

FIG. 3 is a fragmentary view of a basic tip 126 for installation tip 102. Tip 126 has an open end to a cylindrical catheter. Results shown in the Example below demonstrate that with appropriate hydrogel flow properties, installation tip 126 can provide desirable filing of the uterus, and the configuration and relatively large opening provide for low resistance delivery of the hydrogel. In alternative embodiments, openings from the catheter can direct the hydrogel radially away from the catheter to fill the uterus volume, and for these embodiments, the distal end may be closed.

FIGS. 4A-G shows various installation tip 102 embodiments with radially positioned openings along the circumference of the catheter. As shown, the positioning of the side ports may be symmetrical and opposing, extending around a radius, and/or helical in nature, although a wide range of suitable configurations can be appropriate. It will be understood by one of ordinary skill in the art that the positioning of the ports depicted in FIG. 4 is a 2-dimensional representation of a 3-dimensional installation tip. The side ports as shown are intended to represent patterns could also be present on essentially the full circumference in some embodiments and on additional sections of the circumference in other embodiments. FIG. 4A shows an installation tip with side ports designed as a cross pattern of circular holes. FIG. 4B shows an installation tip with side ports designed as an alternating cross pattern of circular holes. FIG. 4C shows an installation tip with side ports designed as a helical pattern of circular holes. FIG. 4D shows an installation tip with a terminal exit 132 and a cap 134. Fluid 136, such as precursor fluid, is shown exiting under the rim of the cap 134. Cap 134 may contain valves or actuated vents through which fluid 136 passes. FIG. 4E shows an installation tip with side ports configured as a series of longitudinal slits. The ports and/or terminal exits may have holes configured in a square, rectangular, circular, oval, diamond, triangle, or polygon shape or mixtures of shapes and sizes. FIG. 4F shows an installation tip with side ports configured as a checkerboard pattern of square holes. The number, shape, size, and arrangement of side ports and terminal exits can be chosen to provide desired coating patterns of the intrauterine surface for a selected hydrogel precursor while simultaneously being constrained with respect to the catheter maintaining column strength during the insertion event into the uterine cavity. Embodiments of a catheter tip with solely an open distal end as a port are presented in examples below while obtaining excellent filling of the uterine cavity while maintaining the hydrogel substantially in the cavity.

FIG. 4G shows an installation tip with multiple side ports organized in a spiral configuration. From side port 140, the most distal side port, the side port diameters and the spacing between side ports decrease in a proximal direction. In one embodiment relating to FIG. 4G, installation tip 138 has an outer diameter of approximately 0.07 inches and side ports 140 located along the distal portion. In some embodiments, side ports 140 may be located in the most distal about 1 cm or about 3 cm or about 5 cm of installation tip 138. In some embodiments, most distal side port 140 has a length of approximately 0.125 inches and a width of approximately 0.625 inches.

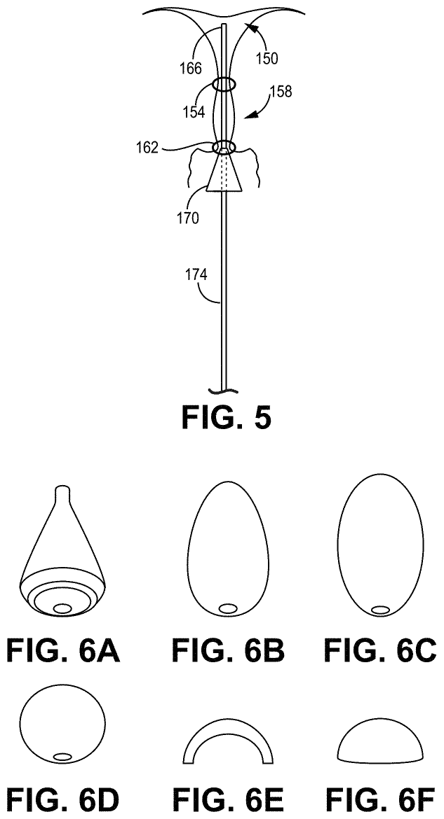

FIG. 5 shows uterine cavity 150, installation tip 166, internal os 154, endocervical canal 158, which is approximately 4 cm in length, external os 162, cap element 170, and catheter 174. Generally, for a particular patient, the health care provider knows with reasonable accuracy the patient's uterine anatomy relating to the uterine length and the length of the endocervical canal such that a closure on the catheter can be adjusted to provide the prescribed distance of the catheter tip from the back of the uterus. The distance from the catheter tip to the back of the uterus following placement of the catheter can be from about 0.25 cm to about 2.0 cm and in further embodiments form about 0.35 to about 1.25 cm. A uterine sound instrument can be used to evaluate the distance sing conventional procedures, and the sound can be held next to the egress limiter to adjust the position of the cap. A person of ordinary skill in the art will recognize that additional distance ranges within the explicit ranges above are contemplated and are within the present disclosure.

As noted above, installation tip 102 desirably presents an atraumatic structure to the patient, which can be characterized by a softness and flexibility. In some embodiments, an atraumatic tip can be formed from elastomers, such as silicone rubbers, rubber, polyisoprene, butyl rubber, mixtures thereof and the like. In additional embodiments, the atraumatic tip can be a second material to the primary catheter shaft material cojoined to the distal end through radiofrequency welding, melting, gluing, or other know methods of attachment. In other embodiments, the atraumatic tip involves a coating added to the end via attachments of different materials or overlay of a coextruded soft flexible material. Materials for the atraumatic tip can be characterizes with respect to their softness using Shore Durometer values and may be possess Shore Hardness 00 values of 20 to 80, with further embodiments in the 00 scale 50 to 70 range. For embodiments in which the transcervical access system may be used to extract fluid, a catheter with a harder tip can be used. A person of ordinary skill in the art will recognize that additional ranges of durometer values within the explicit ranges above are contemplated and are within the present disclosure.

FIG. 6 shows various embodiments of cap element 109 which are shaped to act as a backstop or cervical cap for the prevention of excess material run off during the precursor fluid delivery, as well as reference guide for the placement of installation tip 102. In this context, cap element 109 of egress limiter 106 can be generally highly curved with no sharp points, and it has a radial extended diameter relative to catheter 108. FIG. 6 shows (A) a conical-shaped, (B) a tear drop-shaped, (C) an egg-shaped, (D) a sphere shaped, (E) a flattened hemisphere-shaped, and (F) a dome-shaped cap elements. Embodiments with a dome-shaped cap element can be used to provide a concave seal against the exocervix. The egress limiter design can be selected to meet the desired function to seal the endocervical canal 158. Generally, such an object may be circular, conical or angled one or more surfaces to ensure a secure fit within the outer opening into the cervix, to allow a complete coating of the lumen of the uterus and at least inner portion of the cervix through stabilization of the hydrogel in the enclosed volume as the hydrogel sets sufficiently to avoid loss through the cervix, allowing for a gently pressurized tamponade-like fill of the hydrogel if desired. Thus, cap element 109 can have a radial diameter relative to the catheter axis from about 5 mm to about 1.5 cm and a length along the catheter axis from about 2 mm to about 4 cm and in some embodiments from about 4 mm to about 3 cm. A person of ordinary skill in the art will recognize that additional ranges within the explicit dimensional ranges are contemplated and are within the present disclosure. Cap element 109 can be formed from any appropriate material, such as polymers including, for example, the polymers suitable for the catheter and the installation tip.

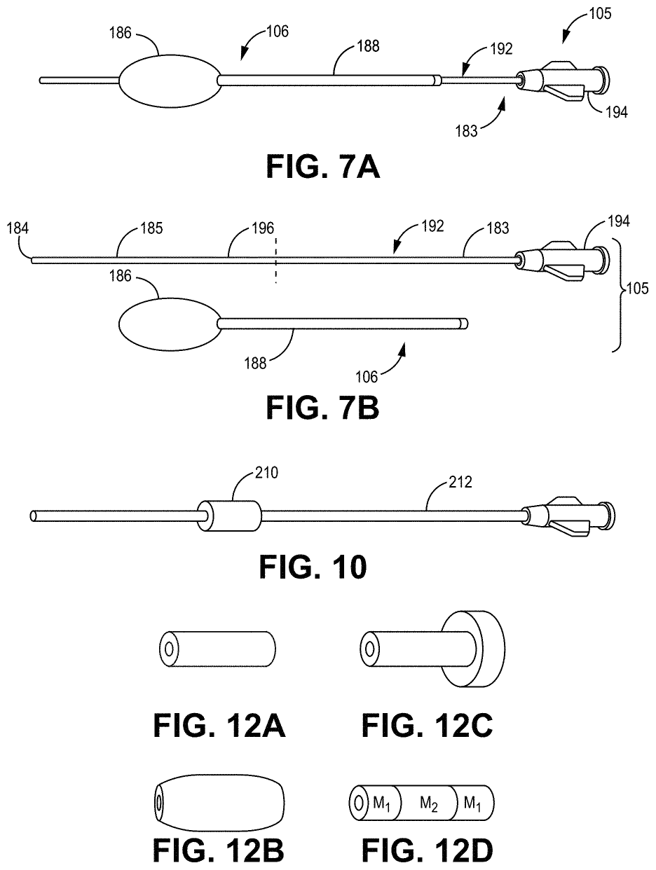

FIG. 7A shows an embodiment in which catheter assembly 105 has catheter 192 and egress limiter 106. Egress limiter 106 comprises cap element 186 and tubular member 188 proximal to cap element 186. Tubular member 188 is supported by the catheter during infusion and is generally less flexible than the length of catheter 192 distal to cap element 186. FIG. 7A depicts the assembled device, while FIG. 7B depicts separated catheter 192 and egress limiter 106. Catheter 192 comprises a connector or hub 194 and a tubular element 196. Length of catheter 192 is designed for insertion into the uterine cavity with cap element 186 against the outer opening into the cervix, referred to as the external orifice or external os. Tubular member 188 can have an adjustable position when assembled to correspond with overlapping all or a portion of the length of tubular element 196 from cap element 186 to connecter 194. Furthermore, tubular element 196 may or may not be uniform along its length with respect to structure and/or composition. As noted above, the catheter tip can be very soft to avoid tissue injury during hydrogel infusion, but a very soft polymer can make manipulation of the applicator more difficult if incorporated along the entire length of tubular element 196. In some embodiments, tubular element 196 comprises a distal port 184 and in some embodiments includes a tip, and a proximal portion 183 that is stiffer than distal portion 185, which is shown with a dashed line to divide these regions. Optional positions for separating a stiffer proximal region is described further below. Proximal portion 183 can be formed from a section of tubing secured over the catheter, a change in material of the catheter, and/or a thickening of the catheter wall. The embodiment with a stiffer proximal section 183 depicted in FIGS. 7A and 7B gives the user greater stability when instrumenting the endocervical canal.

Referring to FIG. 7A, the proximal end of cap element 186 is attached to tubular member 188 to provide egress limiter 106, as shown in a separated configuration in FIG. 7B. In the assembled configuration of FIG. 7A, tubular member 188 provides external stiffening to at least a part of the length of catheter 192 proximal to the cap element. Tubular member 188 also allows for gripping egress limiter 106 to facilitate the procedure. Tubular member 188 can have a length from about 5 cm to about 20 cm, in further embodiments from about 6 cm to about 19 cm, and in some embodiments from about 7 cm to about 18 cm. A person of ordinary skill in the art will recognize that additional ranges of length within the explicit ranges above are contemplated and are within the present disclosure. The egress limiter 106 is engaged over part of catheter 192 as shown in FIG. 7A, generally for insertion of the catheter into the patient. The egress limiter 106 allows the user to adjust the position of cap element 186 to give values of the distal catheter length in the ranges as specified above. In some embodiments, tubular member 188 can internally provide a friction interaction with the catheter surface to restrict unintended motion of the positon of egress limiter 106. The user can set the position of egress limiter 106 and maintain the position along the catheter 192 by avoiding unintended sliding of tubular member 188. In other embodiments, the cap element 186 position can be adjusted and/or maintained via a clip, ridges that engage a flange on the mated element, or the like between catheter 192 and egress limiter 106. Since the cap element 186 is fixedly attached to the tubular member 188, the design of tubular member 188 prevents the inadvertent loss of the cap element 186.

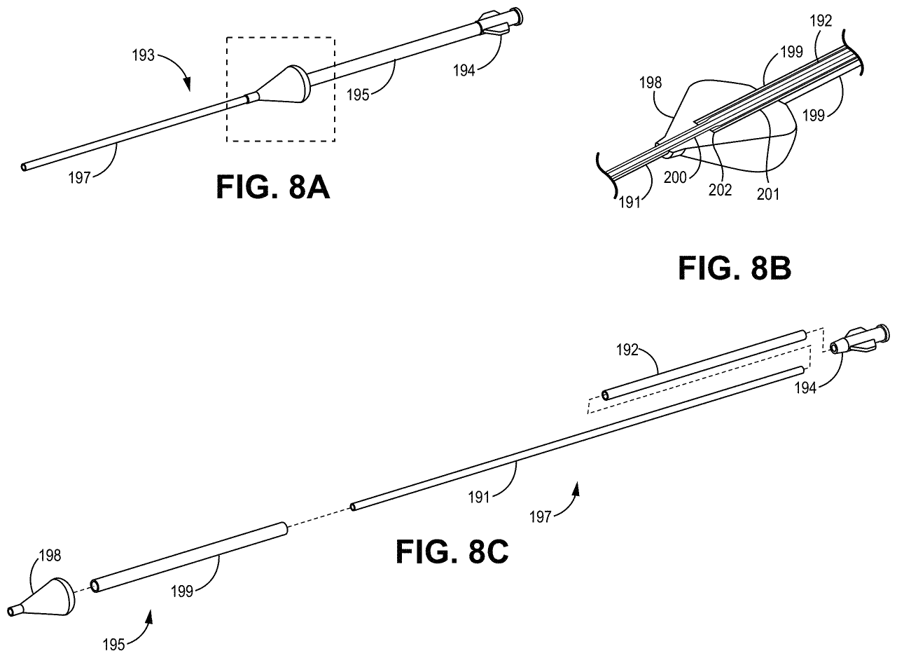

FIG. 8A shows a specific embodiment of a catheter assembly 193 comprising a catheter 197, an egress limiter 195 and connector 194. FIG. 8B shows an enlarged cross-section of the portion of FIG. 8A as indicated by the dashed box. FIG. 8C shows an exploded view of catheter assembly 193. Catheter 197 comprises core tube 191 and overtube 192, both of which engage and are secured to connector 194, which can be a female luer connector or the like. As assembled, catheter 197 has a distal section with a smaller diameter than a proximal section due to the presence of the overtube. Connector 194 can be secured with adhesive, heat bonding, crimping, a combination thereof, or the like, as long as the central lumen remains open. Overtube 192 can be held in place just through securing to connector 194, or also with heat bonding, adhesive bonding or other suitable technique(s) to core tube 191. Overtube 192 provides stiffness to the proximal end of catheter 197 and can provide frictional engagement of egress limiter 195. Overtube 192 can be made thicker and/or with a stiffer material relative to core tube 191. In some embodiments, overtube 192 can have a length from about 5 cm to about 20 cm, in further embodiments from about 6 cm to about 19 cm, and in some embodiments from about 7 cm to about 18 cm. A person of ordinary skill in the art will recognize that additional ranges of length within the explicit ranges above are contemplated and are within the present disclosure.

Referring to FIG. 8C, egress limiter 195 comprises conical cap element 198 and tubular member 199. Conical cap element 198 is attached to tubular member 199 with adhesive or other suitable fastening modality. Referring to FIG. 8B, conical cap element 198 has a cavity with distal diameter 200, proximal diameter 201 and step-down 202. At step-down 202, the cavity diameter within the conical cap element decreases. Step-down 202 provides a mechanical stop to inhibit sliding of egress limiter 195 in a more proximal direction relative to overtube 192. Conical cap element 198 can have a narrow constriction at its distal end to provide a friction grip on catheter 197 that constrains movement of egress limiter 195 along the catheter so that the position can be selected by the medical professional, although friction with overtube 192 can provide itself the desired limits on relative movement of egress limiter 195.

FIG. 9 illustrates an embodiment in which catheter assembly 193 has an egress limiter comprising a cap element 204 that is inflatable, like a balloon, and can be filled with gas or fluid through port 205 to customize the size for the desired fit within the cervix. In some embodiments the fluid can be air, saline or other fluid. Port 205 can have a terminal end that has a Luer connector or the like for attachment with a device to deliver and/or remove fluid. In some embodiments, a syringe or the like can be used to inflate cap element 204 through port 205 to achieve a desired volume, such as through a luer connector. Generally, port 205 connects with a balloon lumen to cap element 204. For example, as shown in a cross-section in the inset of FIG. 9, tubular member 207 may have a balloon lumen 209 while providing for a slidable engagement over catheter 208. In general, a balloon lumen can have any configuration known in the art, such as concentric or non-concentric as shown in the insert of FIG. 9. In some embodiments, after the installation of the hydrogel, cap element 204 may be deflated through port 205 to facilitate easier removal of tubular member 207. Tubular member 207 along with port 205 can be slid off catheter 208 to allow for removal of catheter 208 while holding tubular member 207 with cap element engaged with the outer OS. In some embodiments, the deflation may use a syringe or other negative pressure device.

In some embodiments, it can be desirable to deliver a plug into the cervix to assist in controlling the stabilization of the delivered hydrogel into the uterus and the inner OS of the cervix. Referring to FIG. 10, a plug 210 can have an inner lumen providing for it to slide over catheter 212. Cervical plug 210 can be placed into the cervix prior to delivery of hydrogel precursors into the uterus, and cervical plug 210 can be left in place following removal of the catheter. Cervical plug 210 may swell in contact with moist tissue such that expansion of the cervical plug can help to maintain the position of the cervical plug when the catheter is removed. Alternatively or additionally, the cervical plug can be held in place by the health care professional when the catheter is removed.

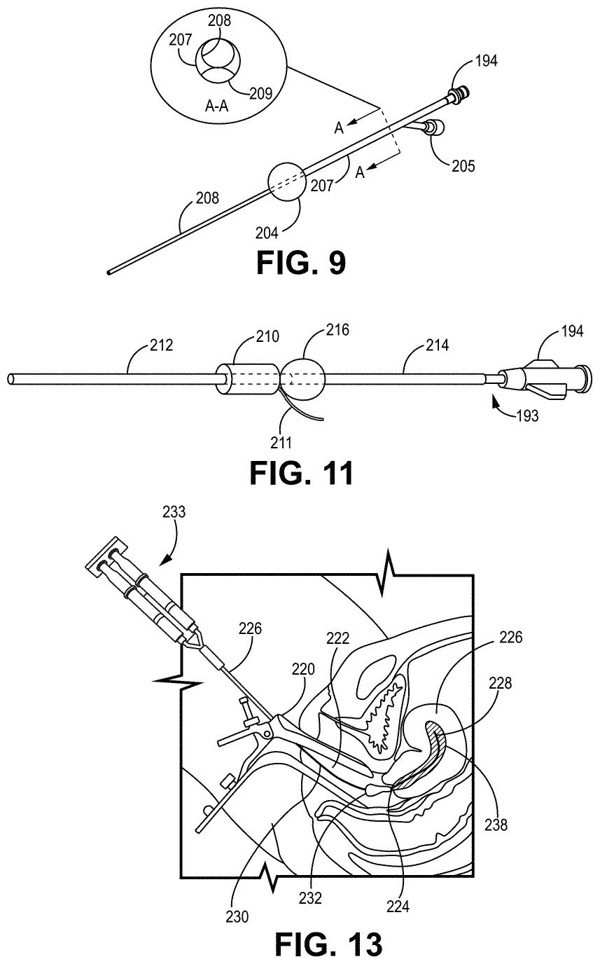

It can be desirable to use both a cervical plug and an egress limiter to control fluid delivery. In particular, the egress limiter can facilitate proper placement of the plug and maintenance of the plug during fluid delivery. Referring to FIG. 11, catheter assembly 193 comprises catheter 212, egress limiter 214, and plug 210. Plug 210 can be placed to abut cap element 216 to anticipate appropriate placement of cervical plug 210 when cap element is placed at the outer OS of the cervix. Cervical plug 210 can be connected to a tether 211 to facilitate removal of plug 210 if desired.

Cervical plug 210 can have a selected shape based on its purpose. FIG. 12 depicts some representative examples of plug shapes, but these are not limiting. Any reasonable shape can be selected if it meets the purpose of impeding expulsion. FIG. 12 A shows a cylindrical shape, which can conform to a natural configuration if it swells. FIG. 12B depicts an oblong shape for plug 210, and this shape may present some restriction to insertion past the outer OS of the cervix, but then its shape helps to keep it in position. FIG. 12C depicts a shape with a capped end that is shaped to engage the inner OS of the cervix similarly to a capping element, such that the capped end can guide insertion of the catheter to a desired depth without a separate egress limiter. The capped end can facilitate removal of a cervical plug at a later time, if desired. FIG. 12D depicts a cervical plug comprised of a distinct material M₁ at the ends versus a material M₂ in the center, where M₂ may swell while M₁ may swell less, or vice versa. In general, the cervical plug can be any reasonable shape that resists expulsion.

Cervical plug 210 may or may not be biodegradable. If the cervical plug is not biodegradable, the cervical plug can be removed by the patient or a health care professional at some appropriate time in the future, such as through the use of tether 211, see FIG. 11. Similarly, cervical plug 210 may or may not swell significantly. Swelling embodiments can be achieved using hydrogels. The cervical plugs are preformed and can be dried into a xerogel for delivery, or they can be delivered in a slightly hydrated form to provide desired elasticity. Biodegradable and non-biodegradable hydrogels and other polymers are described above and are applicable to use for the plug. In some embodiments, the cervical plug following hydration, if applicable, can have a length from about 1.05 cm to about 4.0 cm and in further embodiments from 1.25 cm to 3.5 cm and can have an average diameter relative to the axis of the central lumen at time of delivery from about 4.5 mm to about 9 mm and in further embodiments from about 5 mm to about 8 mm, and a maximum diameter from about 5 mm to about 12 mm and in further embodiments from about 5.5 mm to about 10 mm. The cervical plug generally is generally preformed, and while a hydrogel can provide desirable features other soft materials can be suitable. The swelling from an initial state, which can be positive or negative, can range from −50 wt % to about 1000 wt %, in further embodiments from about −25 to about 300 wt %, and in other embodiment from about −10 wt % to about 200 wt %. Swelling is evaluated after 24 hours in a buffered saline solution relative to the cervical plug as delivered. A person of ordinary skill in the art will recognize that additional ranges within the explicit ranges of cervical plug dimensions and swelling above are contemplated and are within the present disclosure.

### Procedures

As noted above, the transcervical access system with the catheter system described herein can be used effectively for the delivery and/or removal of fluids from the uterine cavity. The transcervical access system in it various embodiments described above is particularly effective for the delivery of hydrogels. Therefore, there is extensive discussion on the delivery of hydrogels. Various applications for the delivery of other fluids are also described. Removal of fluids can be a specific goal or ancillary to other procedures, such as removing fluid prior to hydrogel delivery or removal of fluid delivered into the uterine cavity following use of the fluid.

FIG. 13 illustrates a procedure in which the hydrogel is installed into the uterus transcervically. Vaginal speculum 220 is inserted into vagina 222 to dilate vagina 222. The installation tip 228 of catheter 230 is guided through vagina 222 and cervix 224 and into uterus 226. Installation tip 228 is flexible at the distal end which allows installation tip 228 to adjust to the shape of uterus 226 and reduce risk of trauma. Cap element 232 is positioned a distance from the distal end of installation tip 228 to limit depth of insertion of installation tip 228 into the uterus. In one embodiment, cap element 232 is adjusted to an appropriate distance from the distal end of installation tip 228 so that the medical practitioner can comfortably operate the syringe when installation tip 228 is positioned with cap element 232 adjacent the opening into the cervix with the tip spaced an appropriate distance from the back wall of the uterus. In some embodiments, cap element 232 is part of an adjustable egress limiter (see FIG. 7B, element 190). In some embodiments, catheter 230 with cap element 232 is installed into uterus 226 by the practitioner without syringe assembly 233 attached, then syringe assembly 233 is attached to catheter 230, such as via luer connector 226, in a subsequent step. In some embodiments, a regular empty syringe can be first placed on luer connector 226 to withdraw fluid from the uterine cavity, and following completion of this fluid removal, the syringe can be removed and replaced with syringe assembly 233. Such a stepwise procedure may facilitate single user insertion and application. As discussed above, for appropriate embodiments, the cap element may be adjusted and/or fixed in a position along the catheter length by manually adjusting the proximal end of the egress limiter. When positioned adjacent the opening into the cervix, cap element 232 may function to plug the uterine cavity for through filling and coating with hydrogel. This allows the practitioner a means to inhibit leakage of instilled hydrogel at the external cervical os. Additionally, for some embodiments, cap element 232, if inflatable, may be further adjusted to fit within the cervix by filling with fluid, such as gas or liquid.

For any embodiment of cap element, a distance marker along the catheter can help to position cap element 232 at the proper location for inflation. After installation tip 228 and cap element 232 are placed as desired, syringe assembly 233 is used to introduce one or more precursors and, if applicable, an accelerator solution into the Y connector (optionally containing a static mixing element) to provide a mixed hydrogel forming composition prior to entry into catheter 230. The mixed fluids remain sufficiently fluid until they exit installation tip 228 and then further polymerize and/or crosslink to form a hydrogel 238 that occupies the uterine cavity. In some embodiments, injection is continued without stopping until completed so as to prevent plugging of the catheter 230 and/or the installation tip 228 due to the hydrogel formation. In some embodiments, syringe assembly 233 comprises a plunger cap to facilitate the appropriate volume ratio dispensing from the two syringes.

An outline of procedures particularly suitable for the hydrogel compositions and applications with transcervical delivery into a uterus are presented below. As noted above, the hydrogel precursors and any ancillary compositions can be delivered with 1, 2, 3 or more reservoirs, such as syringes. Compositions may or may not be pre-mixed prior to loading into the syringes prior to and shortly before use. In some embodiments, syringes are filled remotely for use, or the syringes can be filled for use from one or more stock solutions without combination of compositions, but with allowance for adjustment of volumes. In general, the volume of infused liquid, such as hydrogel precursors, can be from 2 cc to 30 cc, in further embodiments from 3 cc to 20 cc, and in some embodiments from 5 cc to 12 cc. A person of ordinary skill in the art will recognize that additional ranges of volumes within the explicit ranges above are contemplated and are within the present disclosure. If a single syringe is used for hydrogel precursor delivery, crosslinking can be controlled by a mixing time, through use of external radiation, such as UV light, through contact with water, through body heat from the patient, through pH change, combinations thereof, or the like. If multiple syringes are used various reactants can be divided appropriately for mixing during delivery. As noted above, hydrogels can be formed from various combinations of one or more precursors that may or may not be polymers themselves and optionally accelerants, catalysts, initiators, activators or the like can be used. In general, any reasonable combination of hydrogel components/reactant can be accommodated with the applicator.

For some embodiments, it is envisioned that the two hydrogel precursors components are delivered from a dual syringe applicator with mixing on the applicator structure prior to delivery onto the catheter. One of the solutions or multiple solutions that are mixed can comprise a visualization agent. If the syringes are not connected to the fittings, in appropriate embodiments, the syringes can be connected up once filled as desired, if appropriate.

The following method of delivery may be advantageously used in a two syringe format to form the intrauterine hydrogel barrier:
1) Prepare each syringe with desired liquid compositions
2) Attach Y connector to each syringe
3) Load both syringes into syringe holder
4) Put plunger cap on syringe ends. Set down.
5) Position egress limiter to correct depth on catheter shaft based on patient's determined anatomy, providing that when the egress limiter is against the cervical external os the distal catheter tip will be roughly below the uterine cavity fundus.
6) Attach syringes with Y connector to catheter.
7) Insert catheter into the uterus via the cervix until the flow limiter is against the cervical external os. (Note that the order of 6) and 7) can be reversed if desired)
8) While applying gentle pressure on the flow limiter against the cervical os, continuously deliver hydrogel through pressing the applicator cap at a relatively constant force until the syringes are empty.
9) Wait up to 10 seconds and gently remove catheter while leaving the egress limiter and/or a cervical plug in place.
10) After the catheter is safely removed, an egress limiter, if used, can be carefully removed, leaving a cervical plug, if present. It may or may not be desirable to wait a short time after removing the catheter to remove an egress limiter.

Once the syringes are prepared, they can be attached to the Y-connector, generally using standard connectors, such as a luer fitting, 2) above. To allow for convenient delivery in some embodiments, the syringes are generally placed in a syringe holder (step 3) above) to allow for handling with one hand, and a plunger cap can be placed to allow for the uniform delivery of liquid simultaneously from both syringes possibly using one hand. The applicator tip can be inserted into the patient to the desired depth, which may be marked with a cap element or the like. If desired the applicator tip can be placed before the syringes are fully connected.

With the applicator tip in place, the uterine cavity can optionally be flushed to remove blood, fluids and potential other materials left from the procedure. For example, a syringe or the like with a flush solution, such as buffered saline or other desired liquid, can be attached to the connector of the applicator tip, for flushing. Although use of the applicator tip can be desirable, flushing can be performed using a different channel, possibly prior to placement of the applicator tip. Flushing can be performed with a selected amount of fluid, or continued until the discharge seems to have cleared the space. Optionally, the syringe can be used to withdraw the flush solution along with any other material from the patient.

When ready for the delivery of the hydrogel precursors into the uterine cavity, the Y-connector can be attached to the connector of the applicator tip, step 6). In alternative embodiments, if the applicator tip is not used for flushing, the Y-connector can be attached to the applicator tip prior to placement of the applicator tip into the patient. The hydrogel precursors are then delivered into the patient, step 7). The syringe cap generally is pushed relatively continuously so that excessive crosslinking does not take place in the applicator tip, although strict continuous delivery is not needed. The rate of delivery can be approximately constant, but again this is not required or even necessarily desired if the force for delivery changes as the cavity fills. In some embodiments, it is desirable to begin delivery of the hydrogel prior to the pot life exceeding 60 minutes. For alternative hydrogel formulations, this time may be altered, and in some embodiments, the hydrogels can be stable for a reasonable shelf life significantly longer than procedure times.

The fluid delivery can be continued until pressure from the uterus pushes back against the cap element. The push back would suggest that the uterine cavity is full of fluid. When the cavity is full, injection may be stopped, or a selected amount of overfill is provided to generate a gentle pressure resulting in a tamponade-like fill within the uterine cavity. This may be desirable in procedures involving intrauterine resections, which could leave open venous channels continuing to bleed post surgery. After stopping the delivery, it is desirable to wait a short period to allow for crosslinking and gelation to take place. After waiting for a reasonable period of time, such as at least 10 seconds and less than 5 minutes, the applicator tip is removed, step 9). Generally, the egress limiter is kept in place with cap element in position so that removal of the catheter does not pull significant hydrogel with it. If a cervical plug is used, this is also left in place. Once the catheter is removed, the cap element can be carefully removed also, leaving behind a cervical plug, if used. With sufficient crosslinking, little if any hydrogel should be lost from the uterine cavity. The completeness of the hydrogel delivery can be confirmed using ultrasound.

For inter-uterine applications, the hydrogel systems can be suitable for transcervical delivery, and the hydrogel can function as a tamponade as well as a material to reduce or eliminate adhesion formation. The design of the hydrogel properties to facilitate these functions are described herein, and the delivery procedure using the applicators is described next.

With properly selected hydrogel properties, it was observed that the hydrogel conformally filled the uterine space. It was also observed that the cornua was filled to the tubal ostium while the fallopian tubes remained clear of hydrogel. The use of an overlow limiter and/or a cervical plug helps to reduce the desirability to withdraw the catheter during the injection event to prevent tunneling or removal of the barrier on device exit.

The catheter length, inner diameter, outer diameter, and materials vary depending on the access requirements, and the following discussion is generally applicable to any of the procedures described herein unless specifically indicated otherwise. The catheter including the installation tip should be of a size appropriate to facilitate delivery, to have a low profile, and cause acceptably low trauma when inserted and advanced to a treatment site. In an embodiment suitable for forming hydrogel implants in the uterus, the installation tip has a distal outer diameter from about 1 mm to about 3 mm to allow delivery through the cervix. The proximal outer diameter of the catheter can be from about 2 mm to about 6 mm, in further embodiments form about 2.5 mm to about 5 mm, and in additional embodiments form about 2.5 mm to about 4.5 mm. The catheter length from the distal tip to connector can be from about 14 cm to about 30 cm, in further embodiments from about 15 cm to about 28 cm and in other embodiments form about 16 cm to about 26 cm. In some embodiments, the catheter OD should be as small as practical to reduce the size of the removal track after formation of the crosslinked gel in-utero. In other embodiments, the distal profile of the catheter to be placed within the cervix should be no more than 9 Fr, in some embodiments no more than 8 Fr, in additional embodiments from 3 Fr to 7 Fr. A person of ordinary skill in the art will recognize that additional length ranges and diameter ranges within the explicit diameter ranges above are contemplated and are within the present disclosure, such as 6 Fr, 5 Fr, 4 Fr.

While the deployment of the hydrogel is often done without visualization and in a blind fashion, it is possible to add visualization agents, such as microbubbles to enable visualization under ultrasound or by adding a radiopacifying agent to enable visualization under X-ray guidance. In embodiments of particular interest, the hydrogel composition has a color agent to provide for convenient visual observation, as described further in the description of the hydrogels. If desired, the treatment space may be filled or flushed with a solution, such as an inert saline solution, to remove blood and other biological fluids from the treatment space prior to delivering the hydrogel. The applicator described in the figures optionally may include an additional lumen to permit flushing liquids to exit the treatment space. Alternatively, a non-inert solution, such as a solution containing a pharmaceutical agent, may be delivered into the treatment space.

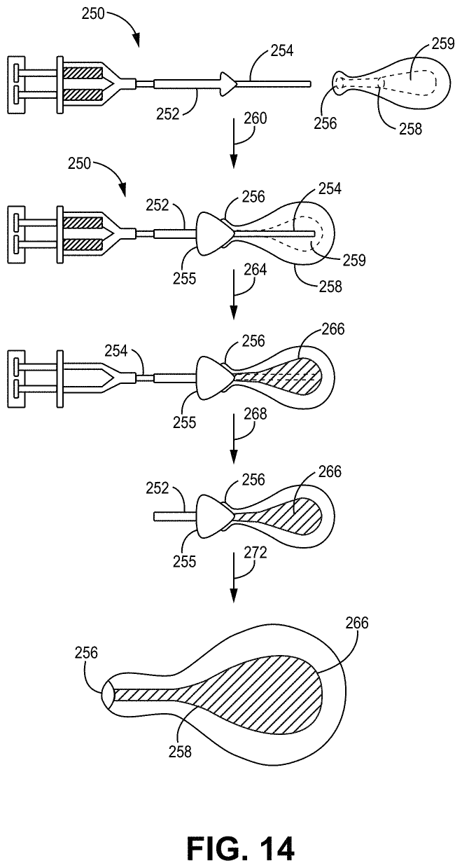

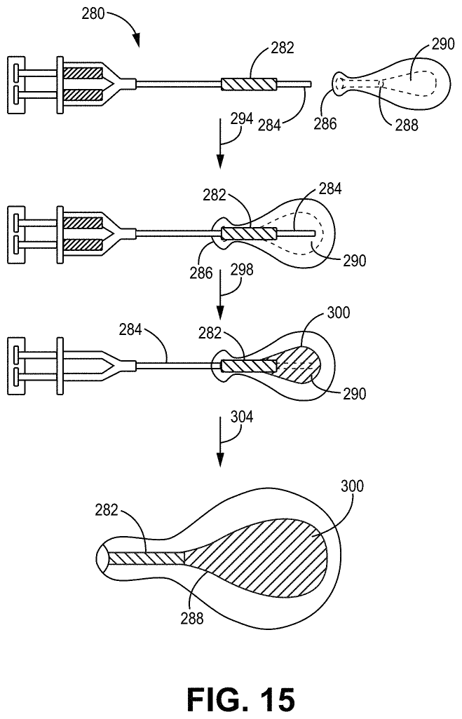

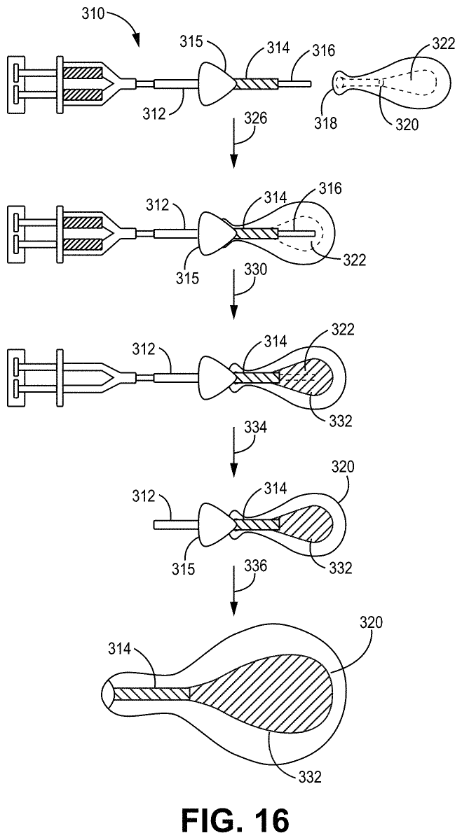

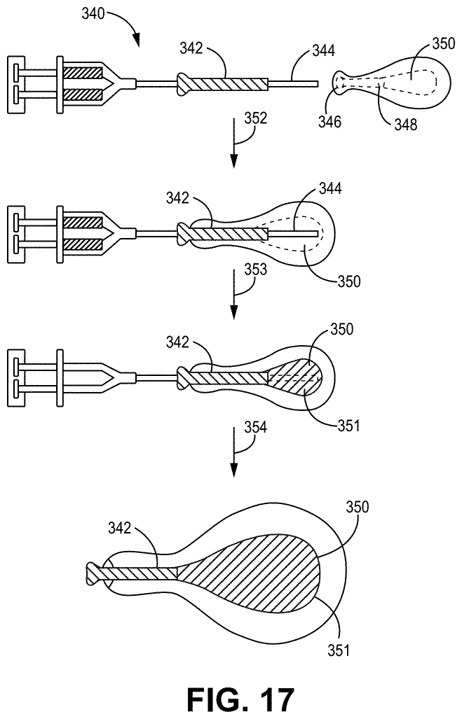

FIGS. 14-17 depict transcervical procedures using various embodiments of the improved procedures based on the transcervical access system designs described herein, with FIG. 14 based on a removable egress limiter, FIG. 15 directed to use of a cervical plug, FIG. 16 directed to use of both a removable egress limiter and a cervical plug, and FIG. 17 directed to a cervical plug with a tether or grip to enable removal of the cervical plug at a selected future time. These figures are presented as procedural flow diagrams with procedural flow progressing from top down.

Referring to FIG. 14, in this embodiment, the transcervical applicator takes the form of applicator 250, which is depicted with egress limiter 252 mounted on catheter 254 aligned for insertion 260 through the cervical outer os 256 past cervical inner os 258 into uterus 258 within uterine cavity 259. Following insertion 260, cap element 255 is positioned at outer os 256 and catheter 254 is within uterine cavity 259, as shown in in the second drawing in FIG. 14. Hydrogel precursor is injected 264 into the uterine cavity to fill the cavity with hydrogel 266. Catheter 254 is then removed 268, as shown in the fourth drawing of FIG. 4, while leaving egress limiter 252 with cap element 255 at outer os 256. Removing 272 egress limiter 252, the last figure of FIG. 14 shows uterus 258 filled with hydrogel 266 extending past inner os of the cervix.

Referring to FIG. 15, the first image at the top depicts an applicator 280 with a cervical plug 282 mounted on catheter 284 positioned for insertion through the outer os 286 of the cervix into uterus 288 within uterine cavity 290. Following insertion 294, the second image of FIG. 15 shows catheter 284 with its tip in uterine cavity 290 with cervical plug 282 within the cervix past the outer os 286. Following injection 298 of hydrogel precursors into the uterine cavity, hydrogel 300 fills uterine cavity 290 up to cervical plug 282. Then catheter 284 is removed 304 from uterus 288 leaving cervical plug 282 in place.

Referring to FIG. 16, the top image depicts an applicator 310 with a egress limiter 312 and a cervical plug 314 distal to the cap element 315 of egress limiter 312 mounted over catheter 316 positioned for insertion into outer os of the cervix into the uterus 320 for placement of the catheter tip in the uterine cavity 322. Following insertion 326, the second image of FIG. 16 shows the tip of catheter 316 in uterine cavity 322 with cervical plug 314 in the cervix and cap element 315 positioned at the outer os of the cervix. Following injection 330 of hydrogel precursors, the third image of FIG. 6 depicts hydrogel 332 within uterine cavity 322 up to cervical plug 314. Following removal 334 of catheter 316 from uterus 320, hydrogel 332 fills the uterus, cervical plug 314 is in place within the cervix, and egress limiter 312 is in position with cap element at the outer os of the cervix. Following removal 336 of egress limiter 312, the last image of FIG. 16 depicts uterus 320 filled with hydrogel 332 with cervical plug 314 still in place.

Referring to FIG. 17, the upper image depicts an applicator 340 comprising a capped cervical plug 342 on catheter 344 positioned for insertion through outer os 346 of the cervix leading into uterus 348 within uterine cavity 350. Upon insertion 352, the second image depicts the tip of catheter 344 in uterine cavity 350 with cervical pug passing through the cervix with the capped end at the outer os of the cervix. Upon the delivery 353 of hydrogel precursors shown in the third image of FIG. 17, hydrogel 351 fills uterine cavity 350. Upon removal 354 of catheter 344, the fourth image of FIG. 17 depicts hydrogel 351 filling uterine cavity 350 with capped cervical plug 342 remaining in place.

As noted above, the transcervical access systems described herein can be used more generally for the delivery of fluids. For example, the transcervical access systems can be used for the delivery of saline as a part of a sonohysterography procedure. If desired, a different tip structure, such as placement of infusion port(s) can be selected in appropriate positions. With the transcervical access systems described herein, a practitioner can have various options for infusion of fluids, such as saline. For example, after infusion of the liquid, the catheter can be removed to leave the limiter in place, which may be easier to maintain in position than the full catheter assembly. In additional or alternative embodiments, therapeutic liquids can be delivered.

In a comparable configuration, the catheter assembly can be used with suction to remove fluids and/or endometrial tissue for pathology through the examination of the cells.

### Drug Delivery

In many applications, the hydrogel applied in contact with a patient's tissue may contain a biologically active agent. Intrauterine drug delivery pathways offer several potential advantages. First, the uterine and vaginal linings are less prone to localized irritation resulting from depot proximity as compared to buccal or ocular mucosal membranes. Second, the intrauterine enzymatic activity is significantly lower in comparison to a gastrointestinal route. Third, the intrauterine pathway bypasses first-pass metabolic losses found in the oral administration pathway, increasing the bioavailability drugs and potentially lowering the dose required. Also, the uterine cavity provides a cul-de-sac which can be filled, unlike the gastrointestinal tract which has continuous flow. As with any localized delivery device, intrauterine therapeutic targets benefit greatly from improved therapy with reduced systemic effects that typically result from higher dose traditional administration routes. The use of hydrogels formed in situ for drug delivery is described in U.S. Pat. No. 9,125,807 to Sawhney et al., entitled “Adhesive Hydrogels for Ophthalmic Drug Delivery,” incorporated herein by reference. The hydrogels can also be enhanced with respect to imaging, as described in U.S. Pat. No. 8,383,161 to Campbell et al., entitled “Radiopaque Covalently Crosslinked Hydrogel Particle Implants,” incorporated herein by reference.

Crosslinked hydrogel materials advantageously may be used for localized or systemic drug therapy via intrauterine administration. Biologically active agents or drug compounds that may be added and delivered from the crosslinked polymer or gel include, for example: proteins, glycosaminoglycans, carbohydrates, nucleic acid, other inorganic or organic biologically active compounds where specific biologically active agents include but are not limited to: enzymes, anti-infectives, antifungals, anti-inflammatories, antineoplastic agents, local anesthetics, analgesics, hormones, angiogenic agents, anti-angiogenic agents, growth factors, antibodies, neurotransmitters, psychoactive drugs, anticancer drugs, chemotherapeutic drugs, drugs affecting reproductivity, genes, oligonucleotides or combinations thereof. In some embodiments, the class of therapeutics targets disease states singular to women's health; these may be local conditions within the uterus itself, and/or health conditions capable of treatment via intrauterine transmucosal transport into the systemic circulation, such as hormonal therapy for post-menopausal women.

To prepare a crosslinked hydrogel composition, the bioactive compounds described above can be mixed with the crosslinkable polymer precursor prior to making an aqueous solution or during aseptic manufacturing of the functional polymer. This mixture then is mixed with the crosslinker or second precursor solution, such as during delivery, to produce a crosslinked material in which the biologically active substance is entrapped. Functional polymers made from inert polymers like Pluronic, Tetronics or Tween” surfactants may be used in releasing small molecule hydrophobic drugs.

In some embodiments, the active agent or agents are sequestered in a separate phase similarly mixed with a precursor or other reagent, when crosslinker and crosslinkable polymers are reacted to produce a crosslinked hydrogel. This sequestration limits or prevents participation of bioactive substance in the chemical crosslinking reaction such as reaction between the ester group and amine group. The separate phase can also help to modulate the release kinetics of active agent from the crosslinked material or gel, where ‘separate phase’ could be oil (oil-in water emulsion), biodegradable vehicle, and the like. Biodegradable vehicles in which the active agent may be present include: encapsulation vehicles, such as microparticles, microspheres, microbeads, micropellets, and the like, where the active agent is encapsulated in a bioerodable or biodegradable polymers such as polymers and copolymers of: poly(anhydride), poly(hydroxy acid)s, poly(lactone)s, poly(trimethylene carbonate), poly(glycolic acid), poly(lactic acid), poly(glycolic acid)-co-poly(glycolic acid), poly(orthocarbonate), poly(caprolactone), crosslinked biodegradable hydrogel networks like fibrin glue or fibrin sealant, caging and entrapping molecules, like cyclodextrin, molecular sieves and the like. Microspheres made from polymers and copolymers of poly(lactone)s and poly(hydroxy acid) are particularly suitable as biodegradable encapsulation vehicles. The use of microspheres with therapeutic agents for an in situ formed hydrogel is described in published U.S. patent application 2016/0166504 to Jarrett et al., entitled “Hydrogel Drug Delivery Implants,” incorporated herein by reference.

In using the crosslinked composition for drug delivery as mentioned above, the amount of crosslinkable polymer, crosslinker and the dosage agent introduced in the host are selected depending upon the particular drug and the condition to be treated. In one embodiment, a crosslinked regional barrier is formed in situ, for example, by electrophilic-nucleophilic reaction, wherein two mixed precursors are simultaneously instilled in a uterine cavity to obtain widespread dispersal prior to gelation and crosslinking of the regional barrier. A therapeutic agent can be dispersed within the crosslinked regional barrier.

Controlled rates of drug delivery may be obtained with the system of the present hydrogels by degradable, covalent attachment of the bioactive molecules to the crosslinked hydrogel network. By using a composite made from linkages with a range of hydrolysis times, a controlled release profile may be extended for longer durations.

In particular for intrauterine delivery, biologically active agents may include anti-infectives or anti-fungals for the treatment of uterine infections, where effectivity of the agent is improved due to its local target proximity. Certain cases may call for the anti-infective agent to be deployed prophylactically during high-risk procedures or in high risk immunocompromised populations. Anti-inflammatories, such as NSAIDs (such as ibuprofen) or corticosteroids (such as prednisone), are another class of agents that may be used to treat conditions such as endometriosis, without the systemic side effects associated with long-term consumption of these agents. In other embodiments, the application of the hydrogel containing an anti-bacterial or anti-viral as a supplemental barrier to a compromised cervix to prevent preterm birth resulting from infection. A range of antibiotics are known in the art and can be delivered through inclusion in the hydrogels.

Procedures for delivery of agents such as hormones can benefit from local intrauterine delivery, ranging from treatment of endometriosis, contraception, and as hormone replacement therapy (HRT) in post-menopausal women. Oral contraceptive consumption is associated with increased risks in thromboembolisms as well as rates of breast cancer. More benign side effects of oral contraceptive use, such as mood changes, weight gain, intermenstrual vaginal bleeding and spotting, and loss of libido, may lead to inconsistent oral administration or discontinuation, translating into a failure rate for oral contraceptives as high as 5% during the first year of use. On the other end of the life cycle, oral administration of HRT in post menopausal women is associated with an increased risk for coronary heart disease, stroke, and venous thromboembolism, as well as increased risk for breast cancer the longer the treatment lasts.

Intrauterine devices (IUDs) are mechanical devices capable of delivering hormones slowly and directly to the uterus. Mirena, a commercially approved levonorgestrel-releasing intrauterine system, is approved for delivery and effectiveness lasting up to 5 years. IUDs offer the advantages of prolonged local delivery of progesterone or levonorgestrel via depots built into the arms of the T-shaped device. IUDs have clinically demonstrated lower side effects associated with low systemic uptake of the hormone therapeutics, but still risk irregular bleeding, perforation, and bacterial/fungal colonization as a result of the mechanical nature and design of the device.

In one embodiment, the application involves delivery of an in situ forming hydrogel with an excess of hormone amounting to 10, 20 30 up to 50% or more suspended in the premixed hydrogel precursor component of the applicator system. Sustained delivery of the hormone would be achieved through the low solubility of these drugs, allowing for extended delivery directly to the uterus for treatment of conditions such as endometriosis. In HRT, the larger hormone doses suitable for contraception or treatment of endometriosis may have adverse side effects even delivered directly to the uterine space. In other embodiments, where delivery control should be precise, low sustained levels of hormone treatment may be obtained through secondary encapsulation of hormone, and suspension of encapsulated agents into the premixed precursor component of the applicator system for delivery. In some embodiments, the secondary encapsulation may use non-erodible materials to achieve even longer therapeutic delivery times; these non-erodible particles would be released, discharged through normal excretions as the hydrogel matrix breaks down and is resorbed.

Endometrial cancer begins in the layer of cells that form the lining (endometrium) of the uterus. Endometrial cancer is sometimes called uterine cancer. Other types of cancer can form in the uterus, including uterine sarcoma, but they are much less common than endometrial cancer. Treatment regiments for endometrial cancer involve surgical removal of the uterus, fallopian tubes and ovaries. In more progressed stages, radiation therapy combined with chemotherapy and/or hormone therapy may be employed. The local delivery of chemotherapy is used in conjunction with radiation or systemic chemotherapy to improve patient outcomes.

In other embodiments, the application of a hydrogel to the uterine cavity takes advantage of the dense vascularization of the uterus, primarily the uterine vein, for the delivery of agents systemically. Agents delivered via the uterus bypass the first pass effect, where total oral bioavailability of the drug may be reduced due to absorption into the hepatic portal system and metabolization by the liver, resulting in excessive doses to reach therapeutic effect. For some agents, oral delivery is not an option at all due to complete loss of the drug to first pass effect. In other cases, the oral administration poses side effects associated with repeat dosing.

Bisphosphonates, a class of drug used for the treatment of osteoporosis, is associated with gastrointestinal distress, inflammation, and erosions of the esophagus. In one embodiment, the intrauterine application of a hydrogel containing a suspension of bisphosphonate particles or encapsulated bisphosphonate to deliver systemic therapeutic levels using less drug without the side effects associated with oral administration. In women who are post-menopausal, intrauterine drug depots could be used to deliver drugs over extended periods of several months.

In addition or as an alternative to drug delivery through the in situ formed hydrogel, drug delivery can be performed using the cervical plug. Drug delivery using the cervical plug is somewhat analogous to drug delivery through a hydogel punctal plug used in the eye, except for the size differential. Thus, the formation of the drug loaded plug can be adapted from U.S. Pat. No. 8,409,606 to Sawhney et al., entitled “Drug Delivery Through Hydrogel Plugs,” and U.S. Pat. No. 10,617,563 to Jarrett et al., entitled “Coated Implants,” incorporated herein by reference.

### Select Embodiment-Adhesion Prevention

The improved applicators and associated delivery methods can be applicable for a range of purposes, such as drug delivery described in the previous section. In some embodiments of particular interest, a method involves preventing adhesion in a uterus, the method comprising introducing a flowable material into a uterus to from a tamponade along the inner surface of the uterus. The tamponade may be effective to reduce bleeding, providing potential patient benefit through reduction of post surgical adhesion formation by preventing egress of serosanguinous exudate. As described herein, the material may be a hydrogel, and the improved processes described herein provide for convenient, effective and reproducible formation of the tamponade or implant. The material may separate at least two opposing portions of the surface to prevent contact between the two opposing portions of the uterus. The material may substantially fill the uterus to provide effective inhibition of adhesion formation, and the material can further fill the cervix to further inhibit adhesion formation. The material may be applied using a gentle pressurized fill resulting in tamponade against bleeding from surgically resected venous channels. The material may be applied through a flexible catheter with a atraumatic tip. The material may be applied through a catheter as described in in detail for various embodiments above. The resulting application can be visualized under ultrasound during and post-administration, the degree of separation of the tissues is quantifiable and translatable to improvement in adhesion prevention.

The application embodiment employs a visualization agent. The visualization agent is in the visible spectrum, ideally comprising blue or green for visualization against tissue. The visualization agent in the hydrogel system may be used to confirm sufficient filling of the uterine space and confirm onset of material crosslinking. In some embodiments, the application uses FD&C Blue #1 to provide a radiation stable precursor.

The material may comprise a hydrophilic polymer. In some embodiments, the material may comprise a polymer comprising the group —(CH₂CH₂O)—. The material may further comprise a therapeutic agent. The material may be degradable in vivo. The material may be hydrolytically degradable. The material may be degradable in vivo in less than about 14 days. The material may contact the surface for at least about one day. The material may be degradable in vivo in more than about one half day and in less than about 7 days. In some embodiments, the material lasts between 3 and 10 days. For utility in pre-menopausal women, a hydrogel that degrades within 21 days is desirable.

The material may be substantially formed in the uterus. The material may be partially formed outside the uterus and formation of the hydrogel may be completed in the uterus. The material may be formed from at least two chemically distinct precursors that react with each other to form the hydrogel. The at least two precursors may comprise a first precursor having a first functional group and a second precursor having a second functional group, wherein the first functional group reacts with the second functional group to form a covalent bond. The material may be formed from two precursors containing the required functional groups to form covalent bonds but mixed in a single solution, wherein the premixed solution is activated by the introduction of a second solution that accelerates the reaction conditions. The first functional group may comprise an electrophile and the second functional group may comprise a nucleophile. The electrophile may comprise a succinimide ester. The nucleophile may comprise an amine. In some embodiments, the electrophile is a large molecular weight succinimide ester and the nucleophile is a small molecular weight amine such as trilysine. The first precursor can comprise at least three of the first functional group, or at least two, four six, or eight. The second precursor can comprises at least four of the second functional group or at least two, six, or eight. In some embodiments, the material and its application uses a large molecular weight first precursor and a low molecular weight second precursor to allow for pre-mixing.

The material may be formed from at least one precursor that forms the hydrogel upon exposure to an activation agent, such as an accelerator agent. The at least one precursor may comprise a polymerizable functional group that comprises at least one vinyl moiety prior to exposure to the activation agent. The polymerizable functional group that comprises the at least one vinyl moiety may be, e.g., acrylate, methacrylate, methylmethacrylate. The polymerizable functional group may be polymerizable using free radical polymerization, anionic polymerization, cationic vinyl polymerization, addition polymerization, step growth polymerization, or condensation polymerization. The activation agent may be a polymerization initiator or a buffer with an elevated pH.

The material may be formed by at least two polymers with opposite ionic charges that react with each other, a composition of a polymer comprising poly(alkylene) oxide and another polymer that undergoes an association reaction with the polymer comprising poly(alkylene) oxide, a thixotropic polymer that forms the hydrogel after introduction into the uterus, a polymer that from the hydrogel upon cooling, a polymer that forms physical crosslinks in response to a divalent cation, and a thermoreversible polymer. The material may comprise a natural polymer. The material may further comprise a visualization agent. An embodiment is a method of preventing adhesion in a uterus, the method comprising crosslinking at least one precursor to form a hydrogel in a uterus to tamponade a surface of the uterus. The hydrogel may be effective to reduce bleeding. At least one precursor may be dry.

A desirable intrauterine anti-adhesive device is easy to use and delivers a hydrogel composition that persists locally during the main phase of adhesion, is re-absorbable and is biocompatible with no interference with the normal tissue repair process. See, Torres-De La Roche L A, Campo R, Devassy R, et al. Adhesions and Anti-Adhesion Systems Highlights. Facts Views Vis Obgyn. 2019; 11:137-149, incorporated herein by reference. The desired system can persist long enough to meet the time window for healing (3-10 days) but not so long that the adhesion barrier itself is encapsulated as part of the healing response. In the case of preventing intrauterine adhesions, injury to the tissue due to incidental contact during the procedure, or from the procedure itself, results in the loss of basement membrane structures, blood-material interactions, provisional matrix formation, cellular necrosis, and inflammatory responses. These events, in turn, may affect the extent or degree of granulation tissue formation, foreign body reaction, and fibrosis or fibrous capsule development. With implants, the process of organization with the development of fibrous tissue leads to the well-known fibrous capsule formation at the tissue/material interface. The ideal persistence of a resorbable adhesion barrier material is two-fold: the material should persist in significant fashion to provide a suitable barrier to adhesion formation, but not persist such time that an adhesion is formed through fibrous encapsulation of the barrier material itself.

Unlike previous commercial hydrogel adhesion barriers for uterine application, which had a persistence in excess of 4 weeks, exemplified hydrogels described herein use only short persisting windows with benchtop disappearance times approximating less than 14 days. These hydrogels can be formed using a succinimidyl succinate (SS) or succinimidyl glutarate (SG) ester material at various concentrations ranging 7-15%, with in some embodiments a range of 9-11%.

### EXAMPLES

The examples below used a transcervical access system that was supplied with two solutions, one in each of two syringes. The first solution was a first precursor or a mixture of a first precursor and a second precursor. The second solution was a second precursor or an accelerator/catalyst. The solutions were mixed within the system during use with a static mixer, such that the mixed solution had an electrophilic precursor and a nucleophilic precursor. The transcervical access system was effectively as described with respect to FIG. 1B. The electrophilic precursor was chosen from four armed PEG-based precursors having either 20,000 Da or 40,000 Da molecular weights and succinimidylglutarate (SG) functional end groups (4A20kSG or 4A40kSG) or an eight armed PEG-based precursor having a 15,000 Da molecular weight and a succinimidyl succinate (SS) ester functional end group (8A15kSS). The nucleophilic precursor was either trilysine acetate or an eight armed PEG-based precursor having a 20,000 Da molecular weight and primary amine terminal functional groups (8A20kNH2). The concentrations of the precursors in the solution(s) were adjusted to provide delivery of an equimolar ratio of nucleophilic end groups to reactive amine end groups for a given delivery system.

### Example 1: Benchtop Studies

This example illustrates the efficacy of a transcervical access system via a benchtop study with a uterine model.

In this example, a benchtop uterine model with a clam-shell design was used. The uterine model consisted of a uterine cavity shaped mold in each side of a plastic clam-shell case. When closed, the model had a circular opening and a tubular space at one end, which simulated the cervix, and an internal triangular space which simulated the body cavity of the uterine cavity.

In this example, the closed uterine model was prefilled with saline using a syringe or a catheter to simulate residual fluids in the uterine cavity that may be present after a transcervical hysteroscopic procedure. The experimental design allowed for testing the efficacy of the transcervical access system with respect to dilution resistance. A transcervical access system was assembled similar to the image in FIG. 1B. A set of first solutions were prepared as a mixture of an electrophilic precursor and a nucleophilic precursor, with a 1:1 ratio of reactive ester end groups to reactive amine end groups for each formulation. A 1.5 ml aliquot of the first solution in 20 mM monobasic buffer solution, pH 4 was drawn into a first syringe. Into a second syringe, 1.5 ml of a pH 9.9 sodium borate/sodium phosphate dibasic accelerator solution was drawn. In each case, the first solutions were colored with a dilute concentration of FD&C blue #1. The second solutions were uncolored.

The accelerator solution-containing syringe and the polymer precursor solution-containing syringe were attached to the Y connector via luer lock connections. A plunger cap was added to the ends of the syringes to ensure equal deployment of the two syringes. The Y connector containing a static mixing element was connected via a third luer lock connection to a 0.25 inch tube adaptor. The tube adaptor was attached to 0.25 inch ID catheter made of clear silicone tubing (Silastic®). The catheter had an open-ended lumen tip. The location of the cap element was adjusted along the catheter length using the egress limiter such that the tip of the catheter would be positioned at a location near the fundus of the simulated body cavity during the insertion step. The catheter of the catheter system was inserted into the cervical opening of the uterine model until the cap element was firmly positioned against the simulated external orifice of the cervix.

Once positioned, the plunger cap was pressed to simultaneously inject the full amount of the solutions from each syringe into the catheter and then into the saline-filled uterine cavity. The injection itself was made over the course of 2-10 s, and completed in less than 10 s. The insertion, positioning, and injection steps were performed with one-handed operation of the transcervical access system. The hydrogel initially formed within a time frame of several seconds and the saline exited through the mold opening past the cap element. Initial gelation was typically observed within 3-5 seconds as evidenced by auxiliary mold-opening experiments. After injection, the cap element was kept in contact with the external orifice while the catheter was removed from the uterine model. After a few seconds, the egress limiter, which includes the cap element, was removed. A comparison study in which the catheter and the cap element were simultaneously removed from the mold was also performed. Samples were allowed to continue to gel over the course of 5 minutes to ensure full cure.

It was observed that the catheter tip did not clog during the delivery and that the hydrogel did not pull out upon removal of the catheter from the mold. The clam shell mold was opened and the hydrogel was inspected. It was observed that the hydrogel filled the mold, including the cavity of the cervix.

The displacement of the fluid from the mold and the visual inspection of the formed hydrogel indicated that the transcervical access system was able to form a solid, flexible dilution resistant hydrogel which fills the uterine cavity, including the cervix. The transcervical access system was further successful in forming a relatively firm hydrogel having a relatively fast gel time, which contributed to successful model intrauterine retention. The results of this study are significant as they indicate that the transcervical access system could be effectively used in the presence of residual intrauterine cavity fluids to form a hydrogel which is firm enough to separate the uterine walls and not be ejected at the end of the installation procedure. The results suggest that the transcervical catheter system could be effectively used to separate the uterine walls after a procedure that creates tissue damage to allow independent healing of these tissue surfaces and prevent the formation of adhesions. The results also suggest that the installed hydrogel would be resistant to dilution from any remaining intrauterine fluids after hysteroscopic transcervical procedures, such as resection to remove unwanted tissues from the uterine cavity.

### Example 2: Human Peri-hysterectomy Comparative Study

This comparative example illustrates the use of an existing transcervical catheter to delivery hydrogel into a human uterus.

Six human patients were part of this study. For each patient, a modified Cook® Goldstein Sonohysterography Catheter was used. The Cook Goldstein Sonohysterography Catheter has a movable acorn-shaped positioner that can be positioned along the catheter, with ink bands located on the catheter as reference marks. The catheter was connected via a luer lock to a dual syringe assembly as described below. In this study, the Cook Goldstein Sonohysterography Catheter was modified by cutting off the catheter at a location proximal to both the round closed tip and the oval sideport. As modified, the catheter had an open port at the distal tip.

For the study, six female patients were chosen. Patient selection was based first on a determination that a hysterectomy was medically needed for the patient and second on the willingness of the patient to participate in the experimental study. Prior to enrollment in the study, a diagnostic hysteroscopy and ultrasonography was be performed and video recorded to assess endometrial thickness, the cervical canal length, uterine cavity length and width, and both ostia to assure that the subject has no pathology that would make them ineligible for the study.

For each patient, the first syringe was filled with a first solution containing a mixture of 18% (w/v) of an electrophilic precursor with reactive ester ends groups and an amount of a nucleophilic precursor to provide a 1:1 ratio of ester and amine end groups. The second syringe was filled with a second solution containing accelerator buffer salts at pH 9.8. The first precursor solution contained a dilute concentration of methylene blue. The second precursor solution was uncolored. The accelerator solution-containing syringe and the polymer precursor solution-containing syringe were attached to a mixing Y connector via luer lock connections. A plunger cap was added to the ends of the syringes to ensure equal deployment of the two syringes. The Y connector was connected via a third luer lock connection to a 21 gauge tube adaptor. The tube adaptor was attached to 21 gauge catheter made of clear polyethylene tubing. The acorn was adjusted along the catheter length based on the anatomy of each patient such that the tip of the catheter would be positioned at a selected location within the cavity of the body of the uterus during the insertion step.

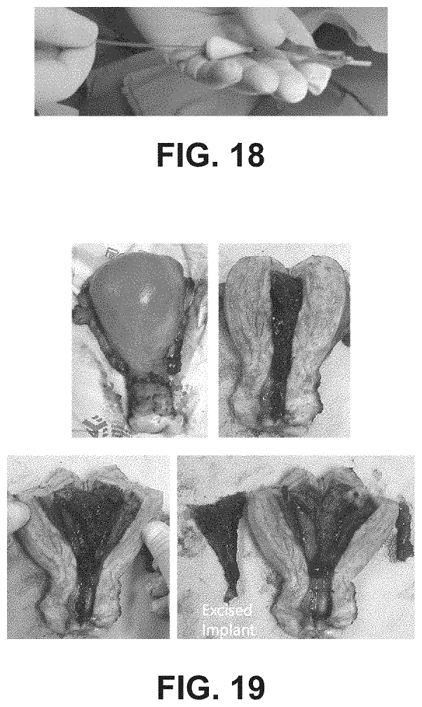

Following the hysteroscopy and ultrasonography, each woman had a radio-frequency non-hysteroscopic endometrial ablation. Following the ablation procedure, the modified Cook Goldstein Sonohysterography Catheter was used to install the hydrogel in the uterus. The catheter of the delivery system was inserted into the cervix through the vagina until the resistance and visible catheter length indicated that the acorn was positioned against the external orifice of the cervix. Once positioned, the plunger cap was pressed to inject a 10 ml quantity of fluid from the syringes into the catheter and then into the uterine cavity. A finger of the surgeon was used to control the acorn. The amount of force applied by the surgeon to the acorn was used to regulate the amount of fluid exiting the cervix during installation. After injection, the catheter with attached acorn was removed from the patient. As shown in FIG. 18, it was observed that the catheter was coated with hydrogel, which was later shown to have compromised the hydrogel implant within the cervical canal. The procedure was modified such that the acorn was held in place via the continued manual pressure of the surgeon's finger while the catheter was pulled through the acorn and removed from the patient. This procedural modification resulted in minimal to no hydrogel being pulled out upon removal of the catheter from the patient. After a period of several seconds of manual pressure on the acorn, the acorn was removed from the patient with a ring forceps. For all of the procedures, it was observed that the catheter tip did not clog during the delivery.

The hysterectomy was then performed to usual standard of care using a surgical method to remove the entire intact uterus. There was no expulsion of the hydrogel during the hysterectomy procedure. The extirpated uterus was sectioned and evaluated for the presence and distribution of the hydrogel implant. All peri-hysterectomy procedures demonstrated fully formed implants. For each patient, it was observed that the intrauterine implant coverage was complete within the body of the uterus and that there was no gel in the fallopian tubes. It was observed that the implant within the cervical canal was more intact for the implants installed with the modified procedure as compared to the unmodified procedure. FIG. 19 shows a series of pathology pictures from one patient in which the modified procedure was used: top left, a removed uterus; top right and bottom left, the removed uterus that has been cut open to reveal the installed hydrogel; bottom right, the cut open uterus with the excised implant. It can be seen that the gel coats the uterine cavity, and the excised implant is a continuous, solid hydrogel that has the shape of the uterine cavity. The excised hydrogel implants were approximately 1 cm in thickness.

While the results of this comparative study were promising, various difficulties were encountered. A first difficulty was the inability of the standard Cook Goldstein Sonohysterography Catheter to deliver the precursor solutions without clogging. This difficulty was partially addressed by cutting off the tip of the catheter, however, the original round, closed tip was removed in the process making introduction of the catheter into the uterus more difficult. A second difficulty was the logistical and procedural issues related to the use of the acorn as a seal. It was observed that the standard Cook Goldstein Sonohysterography Catheter was unable to be used to control the pressure of the acorn against the cervix without additional manual assistance and thereby control hydrogel egress during the procedure. In particular, it was observed that the Catheter was too flexible to transfer sufficient force along its length to the acorn. As a result, control of the acorn generally involved a finger of the practitioner or a finger of an assistant inserted into the vagina to directly contact the acorn. An assistant was further needed to provide tenaculum traction and speculum traction. The process required a practitioner and an assistant. Another difficulty was the pulling out of the hydrogel upon removal of the catheter, as discussed above. With the modified procedure, pulling the catheter through the acorn was difficult since the acorn had a relatively firm fit onto the catheter, the firm fit being part of the design of the Catheter intended to prevent inadvertent slipping or loss of the acorn during a procedure. In such a case of loss, a ring forceps is advised for retrieval of the acorn. In the case of the Catheter used with the modified procedure of pulling the catheter through the acorn and leaving the acorn behind to act as a seal, the procedure also required the removal of the acorn with a forceps. This comparative example highlighted the importance of a catheter system that could be more conveniently and more effectively operated by a surgical practitioner with one hand and without the need for forceps and without the need for an assistant. The transcervical access system described above rectifies these problematic procedural issues and provides more complete hydrogel filling of the uterine cavity, especially at the cervical canal.

### Example 3: Ex-vivo Uterus Benchtop Study

This example illustrates the efficacy of a transcervical access system to deliver hydrogel to a human uterus via an ex-vivo uterus benchtop study.

In this example, an excised human uterus was obtained according to standard medical research protocols. The weight of the ex-vivo uterus was 101 grams.

A transcervical access system similar to the image in FIG. 1B was used. Each syringe had a volume of 10 ml. A set of first solutions were prepared as a mixture of an electrophilic precursor and a nucleophilic precursor, with a 1:1 ratio of reactive ester end groups to reactive amine end groups for each formulation. A 5 ml aliquot of the first solution in 20 mM monobasic buffer solution, pH 4 was drawn into a first syringe. Into a second syringe, 5 ml of a pH 9.9 sodium borate/sodium phosphate dibasic accelerator solution was drawn. In each case, the first solutions were colored with a dilute concentration of FD&C blue #1. The second solutions were uncolored.

A uterine sound (Integra LifeSciences, product number 30-6000) was used to determine the fundal depth of the ex-vivo uterus. Then the uterine sound was placed along the assembly of catheter 108 and egress limiter 106. The position of the cap element of the egress limiter was adjusted along the catheter using the uterine sound as a guide to provide an approximately 1 cm spacing between distal end of installation tip 102 and the fundus during use of the transcervical access system. The catheter and egress limiter were connected to the Y-connector and syringe assembly via a luer fitting. The catheter was inserted into the uterus until the distal portion of the cap element entered the cervical canal and the proximal portion of the cap element was pressed against the external orifice of the cervix. A forceps was used to grasp the lip of the cervix to provide resistance during the insertion process. The system was held by syringe holder 118 and firm pressure between the cervix and the cap element was applied while the plunger was pressed to fully deploy the hydrogel precursors from the two syringes. Next, the catheter was pulled out from the uterus, leaving the egress limiter against the external orifice of the cervix. After approximately 2 seconds, the egress limiter was grasped by support sheath 103 and cap element 109 was pulled away from the cervix. There was no evidence of hydrogel precursor or hydrogel being expelled from the uterus. The uterus was again weighed and determined to be 108 grams. The increase in weight after the installation of the installed hydrogel was 7 grams.

Immediately following, the uterus was sectioned along the sagittal plane. A continuous hydrogel was observed that completely filled the uterine cavity including the cervical canal. The solid hydrogel was removed, and it was noted that it held its shape after removal. The uterus was further evaluated by cutting into the fallopian tubes to check for hydrogel. There was no hydrogel found in the fallopian tubes. The results of this study showed that the transcervical access system was effective at delivering hydrogel to a human uterus to form a hydrogel which fully filled the uterine cavity and was firm enough to separate the uterine walls and not be ejected at the end of the installation procedure. Furthermore, the hydrogel did not enter into the fallopian tubes.

### CITED TECHNICAL ARTICLES (INCORPORATED HEREIN BY REFERENCE TO THE EXTENT INDICATED BELOW)

1. Di Spiezio Sardo, A., Calagna, G., Scognamiglio, M., O'Donovan, P., Campo, R., & De Wilde, R. L. (2016). Prevention of intrauterine post-surgical adhesions in hysteroscopy. A systematic review. European Journal of Obstetrics and Gynecology and Reproductive Biology, 203, 182-192. https://doi.org/10.1016/j.ejogrb.2016.05.050.
2. Hesham Al-Inany. Intrauterine adhesions. An update. Acta Obstet Gynecol Scand 2001; 80: 986-993.
3. Schenker, J. G. (1996). Etiology of and therapeutic approach to synechia uteri. European Journal of Obstetrics and Gynecology and Reproductive Biology, 65(1), 109-113. https://doi.org/10.1016/0028-2243(95)02315-J.
4. Gomel, V. et al.: Pathophysiology of Adhesion Formation and Strategies for Prevention. J. Repro. Med. 41:1, 1996
5. Acunzo, G., et al. (2003). Effectiveness of auto-cross-linked hyaluronic acid gel in the prevention of intrauterine adhesions after hysteroscopic adhesiolysis: A prospective, randomized, controlled study. Human Reproduction, 18(9), 1918-1921. https://doi.org/10.1093/humrep/deg368
6. Guida, M., et al. (2004). Effectiveness of auto-crosslinked hyaluronic acid gel in the prevention of intrauterine adhesions after hysteroscopic surgery: A prospective, randomized, controlled study. Human Reproduction, 19(6), 1461-1464. https://doi.org/10.1093/humrep/deh238.
7. Johns D A, et al., Initial feasibility study of a sprayable hydrogel adhesion barrier system in patients undergoing laparoscopic ovarian surgery. J Am Assoc Gynecol Laparosc 10 (3):334-338, 2003.
8. Taskin, O., et al., (2000). Role of endometrial suppression on the frequency of intrauterine adhesions after resectoscopic surgery. Journal of the American Association of Gynecologic Laparoscopists, 7(3), 351-354. https://doi.org/10.1016/S 1074-3804(05)60478-1.
9. diZerega, G. S.: Use of Adhesion Prevention Barriers in Ovarian Surgery, Tubalplasty, Ectopic Pregnancy, Endometriosis, Adhesiolysis, and Myomectomy. Curr. Opin. Obstet. Gynechol. 8:3, 1996.
10. Drug Facts and Comparisons. Facts and Comparisons, Publishers, St. Louis Mo. 1996.
11. Taskin, O., Sadik, S., Onoglu, A., Gokdeniz, R., Erturan, E., Burak, F., & Wheeler, J. M. (2000). Role of endometrial suppression on the frequency of intrauterine adhesions after resectoscopic surgery. Journal of the American Association of Gynecologic Laparoscopists, 7(3), 351-354. https://doi.org/10.1016/S1074-3804(05)60478-1.
12. Al-Inany, H. (2001). Intrauterine adhesions: An update. Acta Obstetricia et Gynecologica Scandinavica, 80(11), 986-993. https://doi.org/10.1034/j.1600-0412.2001.801103.x.
13. Diamond, M. P., Daniell, J. F., Feste, J., Surrey, M. W., McLaughlin, D. S., Friedman, S., . . . Martin, D. C. (1987). Adhesion reformation and de novo adhesion formation after reproductive pelvic surgery. Fertility and Sterility, 47(5), 864-866. https://doi.org/10.1016/S0015-0282(16)59181-X.
14. Raziel A., Arieli Sholmo: Investigation of the uterine cavity in recurrent aborters. Fertil Steril 1994; 62: 5, 1080-1082.
15. Schenker, J. G., & Margalioth, E. J. (1982). Intrauterine adhesions: an updated appraisal. Fertility and Sterility, 37(5), 593-610. https://doi.org/10.1016/s0015-0282(16)46268-0
15. March C M. Update: Intrauterine adhesions. Fertil News 1996; Vol. XVIV, No. 1. Forum
16. Taylor, P. J., Cumming, D. C., & Hill, P. J. (1981). Significance of intrauterine adhesions detected hysteroscopically in eumenorrheic infertile women and role of antecedent curettage in their formation. American Journal of Obstetrics and Gynecology, 139(3), 239-242. https://doi.org/10.1016/0002-9378(81)90001-6.
17. Nappi, C., Di Spiezio Sardo, A., Greco, E., Guida, M., Bettocchi, S., & Bifulco, G. (2007). Prevention of adhesions in gynaecological endoscopy. Human Reproduction Update, 13(4), 379-394. https://doi.org/10.1093/humupd/dml061.
18. Nappi, C., Di Spiezio Sardo, A., Greco, E., Guida, M., Bettocchi, S., & Bifulco, G. (2007). Prevention of adhesions in gynaecological endoscopy. Human Reproduction Update, 13(4), 379-394. https://doi.org/10.1093/humupd/dml061.
19. Piredda, A., Marconi, D., Exacoustos, C., Sorrenti, G., Zumpano, A., Szabolcs, B., . . . Zupi, E. (2003). Initial Feasibility Study of an Hydrogel Adhesion Barrier System in Patients Treated by Operative Hysteroscopy for Intrauterine Benign Pathologies. 32° Annual Meeting of the AAGL, Las Vegas, Nov. 19-22, 2003, 10(3), 25-26.
20. Victory, R., Berman, J., Diamond, M., Kruger, M., & Mcneeley, S. (2004). Evaluate the Safety and Efficacy of FlowFil Preventing Postoperative Uterine Bleeding and ThermaChoice Endometrial Ablation: 33° Annual Meeting of the AAGL, San Francisco, Nov. 10-13, 2004, 11(3), 29-30.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understood that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicate.

What is claimed is:

1. A transcervical access system for movement of fluids with easy manipulation, the transcervical access system comprising:
a graspable structure comprising one or more fluid reservoirs and one or more actuators to direct flow from or into the one or more fluid reservoirs;
a catheter comprising a tubular element with a lumen, an outer diameter, an average wall thickness, and one or more distal ports, wherein the catheter engages the graspable structure with a configuration to provide fluid flow through the tubular element of the catheter upon actuation of the actuator; and
an egress limiter comprising a tubular member and a cap element fixedly attached to the tubular member at or near an end the tubular member, the tubular member having an inner lumen with an inner diameter larger than the outer diameter of the tubular element of the catheter such that the egress limiter can slide over the catheter and is removable from the catheter, wherein the tubular member has a length less than the length of the tubular element of the catheter, wherein the position of the tubular member allows for adjustment of a distal catheter length, and wherein the distal catheter length comprises a length from the distal end of the catheter to the distal end of the cap element.

2. The transcervical access system of claim 1 wherein the catheter has a length from about 16 cm to about 26 cm and wherein the catheter has an outer diameter from about 1 mm to about 3 mm at the distal end and wherein the tubular element has a flexible distal end.

3. The transcervical access system of claim 1 wherein the one or more distal port of the catheter is an open-ended port coincident with the distal end of the tubular element, and wherein the catheter has an atraumatic distal tip.

4. The transcervical access system of claim 1 wherein the one or more distal port of the catheter is an open-ended port coincident with the distal end of the tubular element.

5. The transcervical access system of claim 1 wherein the tubular element comprises a stiffening segment with a wall thickness greater than the average wall thickness of the tubular element and an outer diameter smaller than the inner diameter of the inner lumen of the tubular member, wherein the stiffening segment has a length that is from about 30% to about 70% of the length of the catheter, wherein the tubular member has a frictional engagement with the stiffening segment such that the length of the distal catheter length can be set by position of the egress limiter with some resistance to change of the length, and wherein the system is suitable for single-handed manipulation.

6. The transcervical access system of claim 1 wherein the tubular member may be grasped during a single-handed manipulation of the system.

7. The transcervical access system of claim 1 wherein the egress limiter comprises a recessed surface and wherein the egress limiter is attached to the tubular element along the recessed surface.

8. The transcervical access system of claim 1 further comprising a cervical plug comprising a lumen with an inner diameter larger than the outer diameter of the tubular element of the catheter such that the cervical plug can slide over the catheter, and wherein the cervical plug is removable from the tubular element and mounted distal to the distal end of the egress limiter.

9. The transcervical access system of claim 8 wherein the hydrated cervical plug has a length from about 1.0 cm to about 4.0 cm and an average outer diameter from about 4.5 mm to about 9 mm, wherein the cervical plug comprises a preformed hydrogel or a preformed xerogel, wherein the cervical plug swells from −25 wt % to +300 wt % as measured after 24 hours in a neutral buffered saline solution, and wherein the cervical plug is hydrolytically degradable in a period of time that is selected to be from 1 day to 5 weeks.

10. The transcervical access system of claim 8 wherein the cervical plug is biodegradable.

11. The transcervical access system of claim 1 wherein the distal tip of the catheter comprises a polymer having a shore hardness value from about 20 A to about 80 A, and wherein the tubular element and the tubular member together have a stiffness that allows for creating a fluid seal between the cap element and the cervix.

12. The transcervical access system of claim 1 wherein the catheter, the tubular member, and the cap element independently comprise silicone rubber, natural rubber, polyisoprene, butyl rubber, polyethylene, polypropylene, nylon, polyether block amide, polyurethane, polysiloxanes, polyvinyl chloride, polycarbonate, PET, copolymers, or mixtures thereof.

13. The transcervical access system of claim 1 wherein the cap element comprises a conical shape, a tear drop shape, an egg shape, a flattened sphere shape, or a dome shape, and wherein the cap element has a length from about 5 mm to about 3 cm and a width from about 5 mm to about 1.5 cm.

14. The transcervical access system of claim 1 wherein the one or more fluid reservoir comprises a first syringe with a connector and a second syringe with a connector, the system further comprising a Y-branch conduit having a first branch connected with the first syringe and a second branch connected with the second syringe and a mixing chamber connected with the first branch and the second branch, the mixing chamber comprising a mixing structure to provide a mixed flow from a distal outlet, wherein the catheter is connected to the distal outlet of the mixing chamber such that mixed fluid flows through the tubular element of the catheter, and wherein the one or more actuator comprises a plate that operably connects to the plungers of the syringes to provide for simultaneous advancement of the plungers.

15. The transcervical access system of claim 14 wherein the mixing structure comprises a static mixer.

16. The transcervical access system of claim 14 wherein the mixing chamber is further connected with multiple ports.

17. A transcervical access system for intrauterine movement of fluids with easy manipulation, the transcervical access system comprising:
a graspable structure comprising one or more reservoirs and one or more actuators to direct flow from or into the one or more fluid reservoirs;
a catheter comprising a tubular element with a lumen, an outer diameter, and one or more distal ports, wherein the catheter engages the graspable structure with a configuration to provide fluid flow through the tubular element of the catheter; and
a cervical plug having an inner lumen with an inner diameter larger than the outer diameter of the tubular element of the catheter such that the cervical plug can slide over the catheter and is removable from the catheter, wherein the cervical plug has an outer diameter suitable for placement in the cervix.

18. The transcervical access system of claim 17 wherein the tubular element comprises a stiffening segment with a wall thickness greater than the average wall thickness of the tubular element and an outer diameter smaller than the inner diameter of the inner lumen of the cervical plug, wherein the stiffening segment has a length that is from about 30% to about 70% of the length of the catheter, wherein the cervical plug is removable from the stiffening segment, the stiffening segment and the tubular element are occupied by a solid material, and/or the cervical plug comprises an ovoid shape, and wherein the system is suitable for single-handed manipulation.

19. The transcervical access system of claim 17 further comprising an egress limiter comprising a tubular member and a cap element fixedly attached to the tubular member at or near an end, the tubular member having an inner lumen with an inner diameter larger than the outer diameter of the tubular element of the catheter such that the egress limiter can slide over the catheter and is removable from the catheter, wherein the cervical plug can be mounted distal to the distal end of the egress limiter, wherein the tubular member has a length less than the length of the tubular element of the catheter, wherein the length of the tubular member allows for adjustment of a distal catheter length, and wherein the distal catheter length comprises a length from the distal end of the catheter to the distal end of the cervical plug.

20. The transcervical access system of claim 17 wherein the catheter has a length from about 16 cm to about 26 cm and wherein the catheter has an outer diameter from about 1 mm to about 3 mm at the distal end and wherein the tubular element has a flexible distal end.

21. The transcervical access system of claim 17 wherein the one or more distal port of the catheter is an open-ended port coincident with the distal end of the tubular element, and wherein the catheter has an atraumatic distal tip.

22. The transcervical access system of claim 17 wherein the cervical plug has a length from about 2 cm to about 6 cm and an initial average outer diameter from about 3 mm to about 10 mm, and wherein the cervical plug swells from −25% to +300% as measured after 24 hours in a physiological solution.

23. The transcervical access system of claim 17 wherein the cervical plug is biodegradable.

24. The transcervical access system of claim 17 wherein the cervical plug comprises a preformed hydrogel or a preformed xerogel, wherein the cervical plug is hydrolytically degradable in a period of time that is selected to be from 1 day to 5 weeks.

25. The transcervical access system of claim 17 wherein the cervical plug comprises crosslinked polyethylene glycol.

26. The transcervical access system of claim 17 wherein the cervical plug position can be selected to a particular location along the catheter.

27. The transcervical access system of claim 17 wherein the cervical plug further comprises a therapeutic agent.

28. A method for transcervical movement of a fluid into or out from a patient's uterine cavity, the method comprising:
transferring fluid into or out from a patient's uterine cavity using a catheter system comprising:
a graspable structure comprising a reservoir of a hydrogel precursor and an actuator,
a catheter comprising a tubular element with a lumen, an outer diameter and one or more distal outlets, wherein the catheter is connected to the reservoir with a configuration to provide fluid flow through the tubular element of the catheter and wherein the tubular element has a suitable length for transcervical intrauterine delivery,
and
a blocking structure comprising a lumen with an inner diameter larger than the outer diameter of the tubular element of the catheter such that the blocking structure can slide over the catheter, wherein the blocking structure has been positioned to adjust a distal catheter length, wherein the distal catheter length comprises a length from the distal end of the catheter to the distal end of the blocking structure; and
removing the catheter from the patient while leaving the blocking structure in place to block the fluid from exiting the cervix.

29. The method of claim 28 wherein the blocking structure comprises an egress limiter comprising a tubular member and a cap element fixedly attached to the tubular member at or near the distal end of the tubular member, wherein the tubular member has an inner diameter larger than the outer diameter of the tubular element of the catheter such that the tubular member can slide over the catheter, wherein the tubular member has a length less than the length of the tubular element of the catheter and wherein the cap element has a geometry suitable for sealing the uterine cervix without fully entering the cervical canal, wherein the sealing inhibits outflow of the hydrogel precursor from the uterine cavity during transferring,
the method further comprising after a selected period of time, removing the egress limiter while leaving an in-situ formed hydrogel in the uterine cavity.

30. The method of claim 28 wherein the blocking structure comprises a cervical plug comprising a tubular section with an inner diameter larger than the outer diameter of the tubular element of the catheter such that the cervical plug can slide over the catheter, wherein the cervical plug is placed in the cervix when the hydrogel precursor is transferred into a patient's uterine cavity and wherein the cervical plug remains in the cervix when the catheter is removed.

31. The method of claim 30 wherein the cervical plug hydrolytically degrades in a period of time that is selected to be from 1 day to 5 weeks.

32. The method of claim 30 wherein the blocking structure further comprises an egress limiter comprising a tubular member and a cap element fixedly attached to the tubular member at or near the distal end of the tubular member, wherein the egress limiter has an inner diameter larger than the outer diameter of the tubular element of the catheter such that the egress limiter can slide over the catheter, wherein the tubular member has a length less than the length of the tubular element of the catheter,
the method further comprising after a selected period of time, removing the egress limiter while leaving the cervical plug in the cervix.

33. The method of claim 32 wherein the cervical plug comprises a preformed hydrogel or a preformed xerogel, wherein the cervical plug undergoes swelling from −25 wt % percent to +300 wt % following placement of the cervical plug at least partially in the cervix, and wherein the swelled cervical plug dilates the inner OS.

34. The method of claim 32 wherein the cervical plug is adjacent to the distal end of the egress limiter and wherein removing the egress limiter comprises rotating the egress limiter along its longitudinal axis to release the connection with the cervical plug, leaving the cervical plug at least partially in the cervical canal.

35. The method of claim 28 wherein the transferring and the removing can be performed with one hand.

36. The method of claim 29 wherein the egress limiter has a length suitable to grasp and wherein the method can be performed with one hand.

37. The method of claim 28 wherein the hydrogel forms in-situ to effectively fill the uterine cavity.

38. The method of claim 28 wherein the hydrogel essentially inhibits contact between cervical tissues and wherein the hydrogel persists in the uterine cavity for a period of time selected from about 1 day to about 6 weeks.

39. The method of claim 30 wherein the cervical plug and/or the hydrogel essentially inhibit contact between cervical tissues.

40. The method of claim 28 wherein the hydrogel precursors form a hydrogel within about 1 second to about 6 seconds after the transferring and wherein the hydrogel is selectively formed in the uterine cavity.

41. The method of claim 28 wherein the blocking structure is positioned at a location along the catheter such that the distal end of the catheter is at a selected location from the fundus of the uterine cavity during the transferring.

42. The method of claim 28 further comprising before transferring, evacuating fluids from the uterine cavity using the system without the reservoir of a hydrogel precursor.

43. The method of claim 28 wherein the blocking structure is positioned to inhibit outflow of the hydrogel precursor from the uterine cavity during transferring.

44. The method of claim 28 wherein the transferring is completed in no more than about 30 seconds.

45. The method of claim 29 wherein the selected period of time is from about 1 second to about 15 seconds.

46. The method of claim 28 wherein delivered hydrogel precursors have a volume from about 10 ml to about 30 ml and wherein the delivered hydrogel provides a tamponade effect.

47. The method of claim 28 wherein the hydrogel precursors comprise a coloring agent and wherein the delivering continues until visualization of colored fluid.

48. The method of claim 28 wherein the blocking structure further comprises a therapeutic agent.

49. The method of claim 28 wherein the catheter has an open-ended port coincident with the distal end of the tubular element and wherein the catheter has an atraumatic distal tip.

50. The method of claim 28 wherein the tubular element comprises a thin diameter distal end with an outer diameter smaller than the average outer diameter of the tubular element.
